(19) United States
(12) Patent Application Publication
(10) Pub. No.: US 2017/0055523 A1
(43) Pub. Date: Mar. 2, 2017

(54) EMULSION DISINFECTING, SANITIZING, AND CLEANING COMPOSITIONS MADE WITH ANTIMICROBIAL AGENTS

(71) Applicant: EnviroSystems, Inc., Orlando, FL (US)

(72) Inventors: Paul S. Malchesky, Painesville, OH (US); J. Lloyd Breedlove, Statesville, NC (US); George E. Grignol, Fairview, PA (US)

(21) Appl. No.: 15/352,018

(22) Filed: Nov. 15, 2016

Related U.S. Application Data
(63) Continuation-in-part of application No. 13/970,704, filed on Aug. 20, 2013, now abandoned.
Application No. 13/970,704 is a continuation-in-part of application No. 13/555,799, filed on Jul. 23, 2012, now abandoned.
(60) Provisional application No. 61/512,017, filed on Jul. 27, 2011.

(51) Int. Cl. A01N 25/04; A01N 25/00; A01N 37/12; A01N 33/12; A61L 2/18; A01N 31/08

(52) U.S. Cl. CPC A01N 25/04; A61L 2/18; A01N 31/08; A01N 37/12; A01N 33/12; A01N 25/00

(57) ABSTRACT

Disclosed are exemplary antimicrobial emulsions having hydrophobic and/or hydrophilic antimicrobial agents, surfactants, solubilizing agents, metal chelators and optionally thickening agents. The emulsions have a small particle size and high zeta potential. The emulsions are effective in cleaning, sanitizing, and disinfecting surfaces and are effective in killing a variety of organisms. The emulsions are stable and have a long shelf life. Other example materials are also disclosed.

### CROSS REFERENCE TO RELATED APPLICATION

This application is a continuation-in-part of U.S. patent application Ser. No. 13/970,704 filed on Aug. 20, 2013 which was a continuation-in-part of U.S. patent application Ser. No. 13/555,799 filed on Jul. 23, 2012 which claims priority from U.S. Provisional Application No. 61/512,017 filed on Jul. 27, 2011.

### TECHNICAL FIELD

The exemplary embodiments relate to compositions containing hydrophobic and/or hydrophillic or low water soluble antimicrobial agents in aqueous solutions that may be applied as a liquid, including in a spray, a wipe or gel, a foam and other forms. The compositions are used to disinfect, sanitize, and clean inanimate or animate surfaces.

### BACKGROUND

Aqueous solutions are used as disinfectants, sanitizers, and cleaning agents for the control, removal, kill, and inactivation of microorganisms on inanimate or animate surfaces to prevent their contamination of the surfaces and those that come in contact with these surfaces.

Various solutions as disinfectants, sanitizers, and cleaning agents are prior art available that carry antimicrobial claims and most utilize water soluble chemistries. Low solubility or non-water soluble, hydrophobic antimicrobials are lipid-like in nature and permit interactions with the hydrophobic structures of microorganisms and thereby inactivating them. These hydrophobic antimicrobials are typically not employed because of the difficulties in solubilizing them and preparing stable liquid formulations. Some past work with hydrophobic antimicrobial agents produce initially high particle sizes in the aqueous emulsion. These large particle sizes increase in size in shelf storage. These hydrophobic agents, when used to create an emulsion, the emulsion must be homogenized with significant mechanical force to create smaller particle size in the emulsion. Even with homogenization, the particles have a low zeta potential which results in an emulsion that is not very stable.

There is a long felt need for an emulsion which uses hydrophobic antimicrobial agents in an aqueous medium that has small particle size and high zeta potentials without the need for homogenization.

There is also an increasing awareness and concern that common household type disinfecting, sanitizing, and cleaning agents can have short term and long term toxic concerns and can be associated with adverse health effects. Further there are concerns for many of the agents that they may accumulate and have an adverse impact on the environment. Therefore, there is a need for disinfecting, sanitizing, and cleaning agents based on natural, biodegradable, and sustainable ingredients such as essential oils and naturally derived surfactants/solubilizing agents which would be good for the user and the environment, while not sacrificing efficacy.

### SUMMARY

An exemplary embodiment provides an antimicrobial water soluble or emulsion composition in an aqueous base which is useful for disinfecting, sanitizing, and cleaning surfaces.

Exemplary embodiments use a hydrophobic antimicrobial agent in the antimicrobial emulsion composition.

An exemplary embodiment provides a ready-to-use antimicrobial composition that is safe for personnel using the composition and is environmentally friendly.

An exemplary embodiment provides an antimicrobial composition that is shelf stable and does not require homogenization in its manufacture.

An exemplary embodiment provides that the ingredients used to make the antimicrobial composition are noncorrosive to the equipment used to manufacture the composition.

An exemplary embodiment provides that the antimicrobial emulsion particles have a small particle size and high zeta potential which leads to excellent shelf stability and does not require homogenization in the manufacture of the emulsion composition.

The above are accomplished by an embodiment of an antimicrobial emulsion formulation comprising:

(a) water;

(b) at least one hydrophobic antimicrobial agent selected from the group consisting of (i) a halogen substituted xylenol compound, (ii) a phenolic compound, (iii) an antimicrobial natural or essential oil, (iv) an antimicrobial component from natural or essential oil, and (v) combinations of at least two of (i), (ii), (iii) and (iv);

(c) at least one surfactant, wherein said surfactant is selected from the group consisting of anionic surfactant, amphoteric surfactant, nonionic surfactant, and blends thereof:

(d) at least one solubilizing agent;

(e) optionally, at least one metal chelator; and

(f) optionally, said antimicrobial emulsion formulation may also comprise ingredients selected from the group consisting of pH adjusters, thickening agents, and colorants.

The emulsion can be used as a ready-to-use spray or wipe or can be thickened to a gel or foam form for more concentrated use.

The terms antimicrobial composition and antimicrobial formulation are used interchangeably herein.

In an exemplary embodiment, the antimicrobial agent in the antimicrobial emulsion composition is parachlorometaxylenol (CAS No. 88-04-0).

In another exemplary embodiment, the surfactant in the antimicrobial emulsion composition is selected from the group consisting of alkyl sulfate, alkyl ether sulfate, potassium ricinoleate, alkylglucoside, and mixtures thereof.

In another exemplary embodiment the solubilizing agent in the antimicrobial emulsion composition is selected from the group consisting of low molecular weight alcohols having from 2 to 10 carbon atoms, glycols, terpineols, phenoxetol, and mixtures thereof.

In another exemplary embodiment the metal chelator in the antimicrobial emulsion composition is selected from the group consisting of trisodium ethylene diamine tetraacetic acid, sodium polyphosphate, and mixtures thereof.

In an exemplary embodiment, water is deionized water.

Excellent results have been obtained with an antimicrobial emulsion composition having

(a) from about 97.78 to about 98.87 weight percent deionized water;

(b) from about 0.2 to about 0.24 weight percent parachlorometaxylenol;

(c) from about 0.2 to about 0.4 weight percent potassium ricinoleate;

(d) from about 0.06 to about 0.1 weight percent sodium dodecyl sulfate;

(e) from about 0.2 to about 0.4 weight percent terpineol;

(f) from about 0.03 to about 0.1 weight percent sodium polyphosphate;

(g) from about 0.3 to about 0.6 weight percent isopropyl alcohol;

(h) from about 0.04 to about 0.08 weight percent phenoxetol; and

(i) from about 0.1 to about 0.3 weight percent trisodium ethylene diamine tetraacetic acid.

### DETAILED DESCRIPTION OF EXEMPLARY EMBODIMENTS

Exemplary embodiments will be described. Various modifications, adaptations or variations of the exemplary embodiments described herein may become apparent to those skilled in the art as such are disclosed. It will be understood that all such modifications, adaptations or variations that rely upon the teachings of the present invention, and through which these teachings have advanced the art, are considered to be within the scope and spirit of the present invention.

The methods and compositions of exemplary embodiments may suitably comprise, consist of, or consist essentially of the components, ingredients, elements, steps and process delineations described herein. Embodiments illustratively disclosed herein suitably may be practiced in the absence of any element, process step, or ingredient which is not specifically disclosed herein.

Unless otherwise stated, all percentages, parts, and ratios expressed herein are based upon weight of the total compositions of the exemplary embodiment.

The term “home care products” as used herein includes, without being limited thereto, products employed in a domestic household for surface cleaning or maintaining sanitary conditions, such as in the kitchen and bathroom or any other inanimate surfaces that are in need of being sanitized.

The term “institutional and industrial care” as used herein includes, without being limited thereto, products employed for surface cleaning or maintaining sanitary conditions in schools, hospitals, nursing homes, restaurants, public transportation, industrial facilities, and offices.

The term “sanitizing” as used herein includes, without being limited thereto, products employed for personal care as soaps, cleansing agents and antiseptics on animate objects. The headings provided herein serve to illustrate, but not to limit the embodiments in any way or manner.

### Antimicrobial Agent

The first necessary ingredient of the antimicrobial emulsion formulation of an embodiment is the antimicrobial agent. In one embodiment the antimicrobial agent is a halogen substituted xylenol, with the preferred antimicrobial agent being parachlorometaxylenol (hereinafter referred to as PCMX). PCMX is also known as 4-chloro-3, 5-dimethyl-hydroxy benzene, 4-cholor 3, 5-dimethyl phenol, 4-chloro 3, 5 xylenol, and 4-chloro meta xylenol. PCMX is a chlorine substituted xylenol with a molecular formula of C₈H₉ClO and has a molecular weight of 156.5 with a CAS No. 88-04-0. PCMX's mechanism of antimicrobial action is by the denaturation of proteins and inactivation of enzymes in the microorganisms. Also likely, this agent, as for other phenolic compounds, alters the permeability of the cell membrane that could result in the uncoupling of oxidative phosphorylation, inhibition of active transport, and loss of pool metabolites due to cytoplasmic membrane damage. Compared to phenols, xylenols exhibit increased antimicrobial activity, on the order of from 30 to 60 times more. The chlorine substitution intensifies the antimicrobial potency.

Other antimicrobial agents which may be used include phenols and substituted phenols, triclosan, trichlocarban, other phenolics such as para tertiary amylphenol (PTAP), o-benzyl-p-chlorophenol (BCP), and ortho-phenyl-phenol (OPP). Other antimicrobial agents include benzalkonium chloride, benzethonium chloride, biguanide, and chlorohexidine gluconate.

As mentioned above, the halogen substituted xylenol (PCMX) is the preferred antimicrobial agent, because it is very effective and environmentally friendly. However, one or more antimicrobial agent may be used in combination with another antimicrobial agent.

In another embodiment, the antimicrobial agent is an antimicrobial natural or essential oil, which can be a natural or synthetic version, or components from such oils that are known to be antimicrobial. Natural or essential oils include terpineol, thyme, wild thyme, red thyme, thyme white, thymol, origanum, oregano and a main constituent carvacrol, lemongrass, lemon, orange, lime, lavender and its constituents lavandin and lavandula, tea tree and its constituents including terpinen-4-ol, wintergreen, eucalyptus and its components as 1,8-cineol and eucalyptol, menthol, cornmint, laurel, ziziphora, bay, sweet orange, cinnamon, cinnamon bark, rose, rosewood, clove, peppermint, rose geranium, geranium, meadowsweet, anise, orris, mustard, rosemary, cumin, neroli, birch, Melissa balm, ylang ylang, juniper, sweet fennel, garlic, cajeput, sassafras, heliotrope, pine, pine oils and their derivatives and components, parsley, violet, coriander, citron, citronella, patchouli, bergamot, sandalwood, eugenol, verbenone, geraniol, limonene, fennel, sesame, geraniol, hinokithiol, citral, terpinene, citronellal, citronellol, linalool, anethole, inenthone, carvone, camphor, and mixtures and components from such.

### Surfactant

An exemplary antimicrobial formulation also contains at least one surfactant. In one embodiment the surfactant is selected from the group consisting of anionic surfactant, amphoteric surfactant, nonionic surfactant, and blends thereof. Anionic surfactants include alkyl sulfates such as sodium lauryl sulfate, sodium laureth (sodium lauryl ether sulfate—SLES) sulfate; ammonium lauryl or laureth sulfate, TEA lauryl or laureth sulfate, MEA lauryl or laureth sulfate, potassium lauryl or laureth sulfate, sodium dodecyl sulfate (SDS), sodium octyl/decyl sulfate, sodium 2-ethyl-hexyl sulfate, sodium octyl sulfate, alkyl ethoxylates, alkyl ethoxylate sulfates, alkyl aryl sulfates, alkyl aryl sulfonates, fatty acid soaps, natural acids saponified such as ricinoleate, alkylsulfonic acid salts, fatty alcohol sulfates, sodium xylene sulphonate, ammonium xylene sulphonate, sodium toluene sulphonate, sodium cumeme sulfate and other hydrotropes, alkyl phosphates as lauryl phosphate, sulfosuccinates as disodium lauryl and laureth sulfosuccinates, alphaolefin sulphonate, and alkyl phenol ether sulfate. Anionic surfactants such as derived from natural sources or recognized as GRAS (Generally Recognized As Safe) that are environmentally friendly are preferred.

Suitable amphoteric surfactants includes the general class of alkyl betaines as laurylamidopropyl betaine, oleyl betaine, ether amine oxides as lauryl dimethyl amine oxide, cocoamidopropyl dimethyl amine oxide and phospholipids composed of diester and triester phosphatides. Amphoteric surfactants such as derived from natural sources or recognized as GRAS that are environmentally friendly are preferred.

Suitable nonionic surfactants includes various liner or non-phenol alcohols or fatty acids, ethers of fatty alcohols, octylphenoxy polyethoxyethanol, ethoxylated alcohols, ethoxylated amines, ether amines and ether diamines as cocoamid DEA, cocoamide MEA, esters as ethylene glycol monostearate, ethylene glycol distearate as polyoxyethylene sorbitan esters, polysorbates, linear ethylene oxide/propylene oxide and/or butylenes oxide block copolymers, poly(5) oxyethylene isodecyloxypropylamine, poly (5) oxyethylene isotridecyloxypropylamine, glycols, and amine oxides as long chain alkyls. Preferred nonionic surfactants include polysorbates as Tween 20, 40, or 80, Igepal, Tritons, and glucosides as decyl glucoside, lauryl glucosides, D-glucopyranoside C10 to C16 alkyl oligomer and D-glucopyranoside C6 to C12 alkyl oligomer. These preferred nonionic surfactants readily biodegrade, are environmentally friendly and are gentle.

### Solubilizing Agent

An exemplary embodiment contains at least one solubilizing agent. The solubilizing agent is necessary because many antimicrobials, such as PCMX, are not soluble or very slightly soluble in water. The combination of the surfactant and solubilizing agent allows a stable emulsion to be made.

Suitable solubilizing agents include low molecular weight alcohols such as ethanol, propanol, isopropanol, glycols such as propylene glycol and polypropylene glycols. Ethanol, isopropyl alcohol, and propylene glycol are among the preferred solubilizing agents. Other preferred solubilizing agents include the cyclic terpenes such as pine oils and their components as the monoterpene alcohols, terpineols, or pine oil derivatives and their isomers alpha, beta and gamma. Note that some ingredients can serve more than one function, such as terpineol which serves as an antimicrobial agent and as a solubilizing agent for the hydrophobic antimicrobial agent. For hydrophilic products water or alcohols are also usable as solubilizing agents.

### Water

An exemplary embodiment contains water. In making the ready to use antimicrobial emulsion formulation suitable for later use, deionized water is highly recommended to provide consistent quality. Although city tap water could be used, deionized water is highly preferred.

### Metal Chelators

Various chemical agents are available to chelate or sequester metal ions in water. They are typically organic molecules and are employed to soften water in formulations. Some examples of metal chelators include organic acids, such as citric acid, sodium and potassium salts of ethylene diaminetetraacetic acid and nitrilotriacetic acid, sodium and potassium salts of methyl glycine diacetic acid, and bisphosphonates. Metal chelators can be important because some of the ingredients, particularly surfactants, can contain metal ions. Also, if water other than deionized water is used, a metal chelator may be necessary.

### pH Adjusters

An exemplary embodiment of the antimicrobial emulsion or water or alcohol soluble products have a pH range of from 6 to 9, preferably from 7 to 8.8, and more preferred from about 7.5 to about 8.5. The pH of the product can be adjusted, if necessary, by using mineral acids, mineral bases, organic acids, and amines. The preferred pH adjusters are hydrochloric acid and citric acid. In some formulations, the pH will be in the desired range and will not need to be adjusted. The pH should be tested and adjusted, if necessary to achieve the desired range mentioned above.

### Skin Protectants/Emollients/Moisturizers

Skin protectants include those identified by the US Food and Drug Administration (see H Zhai and H I Maibach, J Cosmet Derm 1:20-23, 2002) and others such as allantoin (also a keratolyic agent), aluminum hydroxide gel, calamine, cocoa butter, dimethicone, glycerin, kaolin, petrolatum, shark liver oil, white petrolatum, zinc acetate, zinc carbonate, and zinc oxide as well as humectants and emollients and moisturizers and conditioners including polyols as derived from natural materials as castor oil and sucrose including polymeric polyols as polyether and polyester polyols, polyglyceryl monoester, polyglycitol hydrogenated starch hydrolysates, collagen and collagen hydrolysates, soy proteins and their hydrolysates, acacia sengal gum, xanthan gum, hydrolyzed casein, hydrolyzed quinoa, d-gamma tocopherol, DL panthenol, Hamamelis Virginiano (Witch Hazel) extract, Quercus Infectoria (Oak) gall extract, hydrolyzed barley protein, hydrolyzed milk protein, wheat amino acids, wheat starch, hydrolyzed wheat protein, comfrey (Symphytum Officinale) extract, panthenol, hydroxyproline, silk amino acids, hydrolyzed silk, hydrolyzed rice protein, jojoba oil, hydrolyzed keratin, Brassica Campestris/Aleurites Fordii oil copolymer, bisabolol, hydrolyzed glycosaminoglycans, sodium hyaluronate, sorbitol, propylene glycol, perfluorodecalin, aloe vera, vitamins A and E, and chamomile and lavender extracts including combinations of the above.

### Other Pharmaceuticals and Skin Builders

In some applications it is desirable to include other pharmaceuticals than antimicrobial such as cosmeceuticals that can treat the skin or penetrate it and skin builders. Included are anesthetics, skin colorants, skin nutrients as vitamins A (retinoids), C, E, K, and B complex and thiamine, biotin, and niacin; minerals as selenium, copper, and zinc; antioxidants as alpha-lipoic acid, DMAE, hyaluronic acid; and essential fatty acids as omega-3 or omega-6.

Some preferred skin protectants, emollients, or moistures are allantoin, glycerin, propylene glycol, and hydrogenated starch hydrolysates.

### Preservatives

These include natural and synthetic agents including benzisothiazolinone, phenoxyethanol, quaternium-15, potassium sorbate, optiphen, phenonip, rosemary oil, citric acid, parabens as methylparaben, butylparaben, ethylparaben, propylparaben, and other p-hydroxy benzoic acids.

In some exemplary arrangements a preservative used is 1,3-dihydroxymethyl-5,5, dimethylhydantoin also known as DMDMH.

### Foamers

Various chemistries may be added to improve the foaming of the formulation. Some include anionic surfactants as sodium lauryl sulfate, amine oxides as lauryl dimethylamine oxide, and cationic surfactants as cetrimonium chloride, dihydroxypropyl PEG-5 linoleaminium chloride, and fluoroaliphatic phosphates and combinations thereof. Some others include PEG 12 dimethicone, silicone polyether copolymer, DEA-C8-18 perfluoroalkylethyl phosphate.

### Thickeners

In some applications it may be desired to provide the antimicrobial emulsion in a thickened or gel form. To thicken the emulsion thickening agents such as sodium chloride, acrylic polymers, carbomers, polysaccharides as starches and vegetable gums, proteins, and polyethylene glycol may be used to achieve the desired thickened emulsion.

Another important advantage of the antimicrobial emulsion of this invention is the stability of the emulsion without the need for homogenization. To enhance stability of the emulsion it is necessary to have a small particle size and a large zeta potential in the emulsion.

In an exemplary embodiment the emulsion has a particle size of less than 300 nm, preferably less than 100 nm and more preferably from about 60 nm to about 80 nm.

A high zeta potential is also important for stability of the emulsion. Zeta potential is a measure of the potential difference between the dispersion phase (water) and the stationary layer of fluid attached to the dispersed particle and represents the degree of repulsion between adjacent, similarly charged particles. A high zeta potential will confer stability, i.e. the dispersion will resist aggregation. A zeta potential of about 25-30 mV (positive or negative) is considered a value that separates low-charged surfaces from highly-charged surfaces. The zeta potential value can be positive or negative. The important point is the number value. That is a zeta potential of −100 mv is greater than −30 mv and thus is preferred. The zeta potential of the emulsion in this invention is negative and a higher number, either positive or negative is considered better for stability. The exemplary emulsion will have a zeta potential greater than −30 mV, preferably greater than −60 mV, and more preferably greater than −90 mV. Excellent stability results have been obtained with zeta potentials in the range of from −90 mV to −105 mV.

The particle size and the zeta potential can be measured on a Malvern Instruments of Southborough, Mass. Zetasizer Nano-ZS instrument.

The absence of the need to homogenize the emulsion results in a more simplified manufacturing process and gives a cost saving.

In an exemplary method, the process to manufacture the ready-to-use antimicrobial emulsion involves adding the various ingredients (antimicrobial agent, surfactant, solubilizing agent, and if necessary, metal chelator) to water and gently stirring the mixture to create the emulsion. In exemplary arrangements once the emulsion is formed it is filtered with a submicron filter prior to packaging. It is known that raw material chemistries can become contaminated with spore formers and other contaminates that can be picked up in the formulation during manufacturing. Filtration is a manufacturing step that is easy to perform on the ready-to-use emulsion prior to packaging that eliminates spore formers and other contaminants and allows for an aseptic fill. The filtration step provides for a higher quality, more consistent, and robust product. The emulsion can be packaged in any suitable container for later use. Suitable containers include but are not limited to glass or plastic containers such as high density polyethylene (HDPE) and preferably such containers have a spray mechanism to facilitate applying the emulsion to surfaces.

An exemplary antimicrobial emulsion is as follows:

(a) from about 0.18 to about 0.28 weight percent of antimicrobial agent, more preferably from about 0.2 to about 0.24 weight percent, with the preferred antimicrobial agent being parachlorometaxylenol (CAS No. 88-04-0);

(b) from about 0.1 weight percent to about 0.8 weight percent of surfactant, more preferably from about 0.25 to about 0.45 weight percent, with the preferred surfactant being a blend of potassium ricinoleate (CAS No. 7492-30-0) and sodium dodecyl sulfate (CAS No. 151-21-3);

(c) from about 0.2 weight percent to about 1.0 weight percent of solubilizing agent, more preferably from about 0.65 to about 0.85 weight percent, with the preferred solubilizing agent being a blend of alpha terpineol (CAS No. 98-55-5), isopropyl alcohol (CAS No. 67-63-0), and phenoxetol (CAS No. 122-99-6);

(d) from about 0.1 weight percent to about 0.8 weight percent of metal chelator, more preferably from about 0.15 to about 0.40 weight percent, with the preferred metal chelator being a blend of sodium polyphosphate (CAS No. 50813-16-6 or 10124-56-8) and trisodium ethylene diamine tetraacetic acid (trisodium EDTA) (CAS No. 150-38-9);

(e) the remainder of the emulsion is water, more preferably deionized water (CAS No. 7732-18-5), water will usually be present from about 97.78 weight percent to about 98.87 weight percent.

An optimized formulation for the antimicrobial emulsion is shown below.

| Ingredient | level (wt. %) |
|---|---|
| parachlorometaxylenol | 0.22% |
| potassium ricinoleate | 0.30% |
| sodium dodecyl sulfate | 0.08% |
| sodium polyphosphate | 0.05% |
| alpha terpineol | 0.27% |
| isopropyl alcohol | 0.44% |
| phenoxetol | 0.06% |
| trisodium EDTA | 0.20% |
| deionized water | 98.38% |

A prior art formulation for an antimicrobial emulsion is shown below.

| Ingredient | level (wt. %) |
|---|---|
| parachlorometaxylenol | 0.20% |
| potassium ricinoleate | 0.24% |
| alpha terpineol | 0.40% |
| isopropyl alcohol | 0.40% |
| deionized water | 98.76% |

Both the exemplary formulation and the prior art formulation shown above were effective as antimicrobial agents. The prior art formulation had to be homogenized and still had a particle size of 400-600 nm (even higher if not homogenized). The zeta potential of the prior art formulation with homogenization was less than about −20 mV. In contrast the optimized formulation of this invention shown above, which was not homogenized, had a particle size of about 76 nm and a zeta potential greater than about −90 mV.

The above data shows that the exemplary emulsion is a much superior emulsion with higher stability and thus longer shelf life. This results from the smaller particle size and higher zeta potential of the exemplary emulsion.

Both the prior art formulation and the exemplary formulation were effective (kill) against a broad group of organism as shown below.

### Organism

### Bacteria

Staphylococcus aureus
Pseudomonas aeruginosa
Salmonella enteric (cholerasuis)
Methicillin resistant Staphylococcus aureus (MRSA)
Vancomycin resistant Enterococcus faecium (VRE)
Campylobacter jejuni
Escherichia coli
Escherichia coli OH157:H7
Listeria monocytogenes
Legionella pneumophilia
Streptococcus pyrogenes

### Mycobacterium

Mycobacterium bovis
Candida albicans
Trychophyton mentagrophytes

### Fungi

Trychophyton mentagrophytes
Candida albicans

### Viruses

Avian influenza A

### Cytomegalovirus

Herpes simplex virus (type 1 or type 2)
Human Hepatitis B (duck HBV as surrogate)
Human hepatitis C (bovine viral diarrhea virus as surrogate)
Human immunodeficiency virus type 1

### Influenza A2

Rhinovirus type 39
Human coronavirus
Canine parvovirus type-2

Additional examples are presented to better illustrate the exemplary embodiments. Particle size and zeta potential were measured using a Melvern Zetasizer. The stability was also evaluated using a salt test. Salt is known to be effective for destabilizing (breaking an oil in water emulsion) an emulsion. The salt test involves using 25 ml of saturated salt water (sodium chloride). The salt water was added to a glass beaker and gently mixed with a magnetic stirrer. One ml of the antimicrobial emulsion was then added to the beaker containing the salt water. Observations were then made as a function of time. If formulations were not as stable an oily film/droplets will separate out and float. If formulations were more stable either no separation occurred or occurred over much longer time. In the examples, several formulations were compared for stability with the prior art formulation shown above. When the term “prior art formulation” is mentioned, it means the formulation shown above as the prior art formulation, which is believed to be the closest prior art.

The examples presented below are not intended to be limiting but rather to better show the importance of the various ingredients in the formulation.

### EXAMPLES

### Example 1

A composition was made with 0.3% potassium ricinoleate, 0.4% isopropyl alcohol, 0.4% terpineol, and 0.2% PCMX with the remainder deionized water. The solution was made by gentle mixing. The pH was adjusted with concentrated hydrochloric acid (HCl) to pH 8.60. The particle size was determined using a Malvern Zetasizer and determined to be 406 nm. By using a salt test this formulation compared well with the prior art formulation.

### Example 2

A composition was made with 0.3% potassium ricinoleate, 0.06% sodium dodecyl sulfate (SDS), 0.4% isopropyl alcohol, 0.4% terpineol, and 0.2% PCMX with the remainder deionized water. The solution was not homogenized. The pH was adjusted with concentrated hydrochloric acid (HCl) to pH 8.6. The particle size was determined using a Malvern Zetasizer and determined to be 475 nm. By using a salt test this formulation showed less separation vs the prior art formulation and suggested that the use of the additional surfactant improved the stability of the formulation.

### Example 3

A composition was made with 0.3% potassium ricinoleate, 0.2% isopropyl alcohol, 0.2% phenoxetol, 0.4% terpineol, and 0.2% PCMX with the remainder deionized water. The solution was mixed gently by inversion. The pH was adjusted with concentrated hydrochloric acid (HCl) to pH 8.6. The particle size was determined using a Malvern Zetasizer and determined to be 311 nm. With homogenization the particle size was 133 nm. By using a salt test this formulation showed less separation vs the prior art formulation and suggested that the use of the second alcohol improved the stability of the formulation.

### Example 4

A composition was made with 0.3% potassium ricinoleate, 0.3% isopropyl alcohol, 0.1% phenoxetol, 0.4% terpineol, and 0.2% PCMX with the remainder deionized water. The solution was mixed gently by inversion. The pH was adjusted with concentrated hydrochloric acid (HCl) to pH 8.4. The particle size was determined using a Malvern Zetasizer and determined to be 219 nm and was 152 after homogenization. By using a salt test this formulation showed less separation vs the prior art formulation and suggested that the reduction of the phenoxetol did not reduce the stability of the formulation.

### Example 5

A composition was made with 0.3% potassium ricinoleate, 0.09% SDS, 0.3% isopropyl alcohol, 0.1% phenoxetol, 0.4% terpineol, and 0.2% PCMX with the remainder deionized water. The solution was mixed gently and then homogenized. The pH was adjusted with concentrated hydrochloric acid (HCl) to pH 8.6. The particle size was determined using a Malvern Zetasizer and determined to be 547 nm and 128 nm after homogenization. By using a salt test this formulation showed less separation vs the prior art formulation.

### Example 6

A composition was made with 0.3% potassium ricinoleate, 0.06% SDS, 0.8% isopropyl alcohol, 0.4% terpineol, and 0.2% PCMX with the remainder deionized water. The solution was mixed gently and then homogenized. In several samples of this composition, the pH was adjusted with concentrated hydrochloric acid (HCl) to pH 6.9-10.0. The particle size was determined using a Malvern Zetasizer post homogenization to be 122-156 nm with the lower pH having the higher particle size. By using a salt test on this formulation the higher pH formulation was less stable and the pH of about 8.5 the most stable, with all more stable than the prior art formulation.

### Example 7

A composition was made with 0.3% potassium ricinoleate, 0.06% SDS, 0.8% isopropyl alcohol, 0.8% terpineol, and 0.2% PCMX with the remainder deionized water. The solution was mixed gently and then homogenized. In several samples of this composition the pH was adjusted with concentrated hydrochloric acid (HCl) to pH 3.8-9.8. The particle size was determined using a Malvern Zetasizer post homogenization to be 174-879 nm with the higher pHs having the higher particle sizes. By using a salt test on this formulation the higher pH formulation was less stable and the pH of about 8.0 the more stable of the formulations studied with all more stable than the prior art formulation.

### Example 8

A composition was made with 0.24% potassium ricinoleate, 0.06% SDS, 0.4% isopropyl alcohol, 0.4% terpineol, and 0.2% PCMX with the remainder deionized water. The solution was mixed gently and then homogenized. The pH was adjusted with concentrated hydrochloric acid (HCl) to pH 8.5. The particle size was determined using a Malvern Zetasizer post homogenization to be 151 nm with zeta potential of −111 mV versus −21 mV for the prior art formulation. By using a salt test on this formulation was more stable than the prior art formulation.

### Example 9

A composition was made with 0.24% potassium ricinoleate, 0.06% SDS, 0.4% isopropyl alcohol, 0.2% terpineol, and 0.2% PCMX with the remainder deionized water. The solution was mixed gently and then homogenized. In two samples of the composition the pH was adjusted with concentrated hydrochloric acid (HCl) to pH 8.5 and 7.0. The particle size was determined using a Malvern Zetasizer post homogenization to be 305 nm at pH 8.5 and 349 nm at pH 7.0. Particle size was 443 nm prior to homogenization. By using a salt test these formulations were more stable than the prior art formulation.

### Example 10

A composition was made with 0.24% potassium ricinoleate, 0.06% SDS, 0.3% isopropyl alcohol, 0.1% phenoxetol, 0.4% terpineol, and 0.2% PCMX with the remainder deionized water. The solution was mixed gently and then homogenized. In two samples of this composition the pH was adjusted with concentrated hydrochloric acid (HCl) to pH 8.5 and 7.0. The particle size was determined using a Malvern Zetasizer post homogenization to be 228 nm at pH 8.5 and 293 nm at pH 7.0. Particle size was 479 nm prior to homogenization. By using a salt test these formulations were more stable than the prior art formulation.

### Example 11

A composition was made with 0.24% potassium ricinoleate, 0.06% SDS, 0.4% isopropyl alcohol, 0.2% terpineol, and 0.2% PCMX with the remainder deionized water. The solution was mixed gently and then homogenized. In several samples of this composition the pH was adjusted with concentrated hydrochloric acid (HCl) from 6.8-8.0. The particle size was determined using a Malvern Zetasizer post homogenization to be 282 nm at pH 6.8, 217 nm at pH 7.4, and 98 nm at pH 8.0. Particle size was 338 nm prior to homogenization at pH 7.4. By using a salt test these formulations were more stable than the prior art formulation and comparable at pH 6.8-8.0 and to the formulation in Example 8 at pH 8.5.

### Example 12

A composition was made with 0.24% potassium ricinoleate, 0.06% SDS, 0.4% isopropyl alcohol, 0.3% terpineol, and 0.2% PCMX with the remainder deionized water. The solution was mixed gently and then homogenized. In several samples of this composition the pH was adjusted with concentrated hydrochloric acid (HCl) from 6.9-8.1. The particle size was determined using a Malvern Zetasizer post homogenization to be 207 nm at pH 8.1, 246 nm at pH 7.5, and 252 nm at pH 6.9. Particle size was 527 nm prior to homogenization at pH 8.8. By using a salt test these formulations were more stable than the prior art formulation and near comparable to the formulation in Example 11 at pH 7.4.

### Example 13

A composition was made with 0.24% potassium ricinoleate, 0.06% SDS, 0.4% isopropyl alcohol, 0.1% terpineol, and 0.2% PCMX with the remainder deionized water. The solution was mixed gently. While adding the water the solution turned cloudy and crystals formed and separated with mixing believed to be due to the low terpineol concentration.

### Example 14

A composition was made with 0.24% potassium ricinoleate, 0.06% SDS, 0.4% isopropyl alcohol, 0.15% terpineol, and 0.2% PCMX with the remainder deionized water. The solution was mixed gently. While adding the water the solution turned cloudy and crystals formed and separated with mixing believed to be due to the low terpineol concentration.

### Example 15

A composition was made with 0.23% potassium ricinoleate, 0.06% SDS, 0.5% isopropyl alcohol, 0.19% terpineol, and 0.27% PCMX with the remainder deionized water. The solution was mixed gently. While adding the water the solution turned cloudy and crystals formed and separated with mixing believed to be due to the low terpineol concentration.

### Example 16

A composition was made with 0.24% potassium ricinoleate, 0.06% SDS, 0.4% isopropyl alcohol, 0.2% terpineol, and 0.2% PCMX with the remainder deionized water. The solution was mixed gently and then homogenized. In several samples of this composition the pH was adjusted with concentrated hydrochloric acid (HCl) to 7.4-7.7. In two versions trisodium EDTA was added at 0.1 and 1.0%. For pH 7.6 without EDTA the particle size was 85 nm, for pH 7.7 with 0.1% EDTA the particle size was 91 nm, and for pH 7.6 with 1.0% EDTA the particle size was 284 nm. Particle size was 527 nm prior to homogenization at pH 8.8. By using a salt test these formulations were more stable than the prior art formulation. In particular the formulations at pHs 7.6 and 7.7 were the most stable.

### Example 17

A composition was made with 0.24% potassium ricinoleate, 0.06% SDS, 0.4% isopropyl alcohol, 0.2% terpineol, and 0.12% hydrogen peroxide with the remainder deionized water. The solution was mixed gently and then homogenized. The pH was adjusted to 8.5. The particle size was 291 nm. Within a day noticeable degassing of the solution occurred.

### Example 18

A composition was made with 0.24% potassium ricinoleate, 0.06% SDS, 0.4% isopropyl alcohol, 0.2% terpineol, and 0.2% PCMX with the remainder deionized water. The solution was mixed gently and then homogenized.

### Example 19

A composition was made with 0.24% potassium ricinoleate, 0.06% SDS, 0.4% isopropyl alcohol, 0.2% terpineol, and 0.2% PCMX with the remainder deionized water. The solution was mixed gently and then homogenized.

### Example 20

A composition was made with 0.24% potassium ricinoleate, 0.06% SDS, 0.4% propylene glycol, 0.2% terpineol, and 0.2% PCMX with the remainder deionized water. The solution was mixed gently and then homogenized. In two samples of this composition the pH was adjusted with concentrated hydrochloric acid (HCl) to 7.3 and 7.4. In two versions trisodium EDTA was not added in the formulation at pH 7.3 and for the formulation at pH 7.4 EDTA was added at 0.1%. For the formulation at pH 7.3 without EDTA the particle size was 240 nm and for the formulation at pH 7.4 with 0.1% EDTA the particle size was 87 nm. The addition of the EDTA significantly lowered the particle size.

Note: For the Example 20 formulations crystallization of the PCMX occurred after refrigeration. In subsequent evaluations it was determined that the crystallization is related to the terpineol concentration and that it must be a minimum of just over 0.2%. In some studies the presence of EDTA appeared to reduce the crystallization.

### Example 21

A composition was made with 0.24% potassium ricinoleate, 0.06% SDS, 0.3% isopropyl alcohol, 0.1% phenoxetol, 0.2% terpineol, and 0.2% PCMX with the remainder deionized water. The solution was mixed gently and then homogenized. In two samples of this composition the pH was adjusted with concentrated hydrochloric acid (HCl) to 7.3 and 7.4. In two versions trisodium EDTA was not added in the formulation at pH 7.3 and for the formulation at pH 7.4 was added at 0.1%. For the formulation at pH 7.3 without EDTA the particle size was 131 nm and for the formulation at pH 7.4 with 0.1% EDTA the particle size was 61 m. The addition of the EDTA significantly lowered the particle size.

### Example 22

A composition was made with 0.24% potassium ricinoleate, 0.06% SDS, 0.3% isopropyl alcohol, 0.1% phenoxetol, 0.2% terpineol, 0.04% sodium polyphosphate, and 0.2% PCMX with the remainder deionized water. The solution was mixed gently and then homogenized. The pH was adjusted with concentrated hydrochloric acid (HCl) to 7.45. Various formulations were produced from this with varying concentrations of EDTA from zero to 0.3%. At pH 7.5 the particle size was 61 nm. Without homogenization and without EDTA the pH was 8.5 and particle size of 43 nm; without homogenization and with EDTA of 0.14% the pH was 8.3 and particle size of 30 nm. By using a salt test these formulations were more stable than the prior art formulation.

Note: The major difference in this formulation was the addition of polyphosphate but leading up to this formulation particle sizes were becoming lower pre homogenization.

### Example 23

A composition was made with 0.24% potassium ricinoleate, 0.06% SDS, 0.3% isopropyl alcohol, 0.1% phenoxetol, 0.2% terpineol, 0.04% sodium polyphosphate, 0.01% ricinoleamidopropyl PG-dimonium chloride phosphate, and 0.2% PCMX with the remainder deionized water. The solution was mixed gently and homogenized. For formulations to which no EDTA was added, 0.05%, and 0.1% EDTA the particle sizes were 70 nm, 66 nm, and 64 nm and pHs were 7.4, 7.4, and 7.4 respectively. For solutions refrigerated no crystallization was noted for storage of greater than 105 days. By using a salt test these formulations were more stable than the prior art formulation.

### Example 24

A composition was made with 0.24% potassium ricinoleate, 0.06% SDS, 0.2% isopropyl alcohol, 0.2% phenoxetol, 0.1% terpineol, 0.04% sodium pyrophosphate, and 0.2% PCMX with the remainder deionized water. The solution was mixed gently. Crystals formed in the process of making the formulation (likely related to the very low terpineol concentration).

### Example 25

A composition was made with 0.24% potassium ricinoleate, 0.06% SDS, 0.3% isopropyl alcohol, 0.1% phenoxetol, 0.26% terpineol, 0.04% sodium polyphosphate, and 0.2% PCMX with the remainder deionized water. The solution was mixed gently and homogenized except for one portion. For the formulation not homogenized and to which the concentration of EDTA was 0.2%, the pH was 7.5 and particle size 71 nm. For portions of this solution with EDTA and homogenized the pH was 7.4 and the particle size 72 nm suggesting that at such a low particle size homogenization did not further reduce it. For a solution homogenized but with 0.4% EDTA the pH was 7.5 and particle size of 61 nm suggesting that increasing the EDTA concentration did not further reduce the particle size significantly. For a formulation of the same composition but with 0.25% PCMX, homogenized, with an EDTA concentration of 0.2% the pH was 7.4 and the particle size was 90 nm and for an EDTA concentration of 0.4% the pH was 7.4 and the particle size was 66 nm. No crystallization was seen in refrigerated samples for greater than 621 days. By using a salt test these formulations were more stable than the prior art formulation.

### Example 26

A composition was made with 0.24% potassium ricinoleate, 0.06% SDS, 0.3% isopropyl alcohol, 0.1% phenoxetol, 0.22% terpineol, 0.04% sodium polyphosphate, and 0.2% PCMX with the remainder deionized water. The solution was mixed gently only. The pH was adjusted with hydrochloric acid (HCl) and EDTA added to 0.2%. The pH was 7.4 and particle size 69 nm. No crystallization was noted in refrigerated samples for greater than 531 days. By using a salt test these formulations were more stable than the prior art formulation.

### Example 27

A composition was made with 0.24% potassium ricinoleate, 0.06% SDS, 0.3% isopropyl alcohol, 0.1% phenoxetol, 0.2% terpineol, 0.04% sodium polyphosphate, and 0.2% PCMX with the remainder deionized water. The solution was mixed gently only. The pH was adjusted with hydrochloric acid (HCl) and EDTA added to 0.2%. The pH was 7.4 and particle size 78 nm. Crystallization was noted in refrigerated samples at 16 days likely related to the lower terpineol concentration. By using a salt test these formulations were more stable than the prior art formulation.

### Example 28

A composition was made with 0.24% potassium ricinoleate, 0.06% SDS, 0.3% isopropyl alcohol, 0.1% phenoxetol, 0.21% terpineol, 0.04% sodium polyphosphate, and 0.2% PCMX with the remainder deionized water. The solution was mixed gently only. The pH was adjusted with hydrochloric acid (HCl) and EDTA added to 0.2%. The pH was 7.4 and particle size 64 nm. Crystallization was noted in refrigerated samples at 29 days likely related to the lower terpineol concentration. By using a salt test these formulations were more stable than the prior art formulation.

### Example 29

A composition was made with 0.24% potassium ricinoleate, 0.06% SDS, 0.3% isopropyl alcohol, 0.1% phenoxetol, 0.25% terpineol, 0.04% sodium polyphosphate, and 0.2% PCMX with the remainder deionized water. The solution was mixed gently only. The pH was adjusted with hydrochloric acid (HCl) and EDTA added to 0.2%. The pH was 7.4 and particle size 68 nm. Crystallization was not noted in refrigerated samples for over 579 days likely related to the higher terpineol concentration. By using a salt test these formulations were more stable than the prior art formulation.

### Example 30

A composition was made with 0.19% potassium ricinoleate, 0.05% SDS, 0.32% phenoxetol, 0.17% terpineol, 0.03% sodium polyphosphate, and 0.16% PCMX with the remainder deionized water. The solution was mixed gently only. The pH was adjusted with hydrochloric acid (HCl). To a portion no EDTA was added and the pH was 7.4 and particle size 71 nm. To another portion EDTA was added to 0.2% EDTA and the pH was 7.5 and particle size 66 nm. Crystallization was not noted in refrigerated samples for over 564 days.

### Example 31

A composition was made with 0.24% potassium ricinoleate, 0.06% SDS, 0.1% isopropyl alcohol, 0.3% phenoxetol, 0.22% terpineol, and 0.2% PCMX with the remainder deionized water. The solution was mixed gently only. The pH was adjusted with hydrochloric acid (HCl). To a portion of the mix no EDTA was added and to another portion EDTA was added at 0.1%. For the non-EDTA portion the pH was 7.4 and particle size 70 nm. For the portion to which EDTA was added the pH was 7.4 and the particle size 63 nm. For each solution no crystallization was noted following refrigeration of the samples for greater than 563 days.

### Example 32

A composition was made with 0.24% potassium ricinoleate, 0.06% SDS, 0.2% isopropyl alcohol, 0.2% phenoxetol, 0.22% terpineol, and 0.2% PCMX with the remainder deionized water. The solution was mixed gently only. The pH was adjusted with hydrochloric acid (HCl). To a portion of the mix no EDTA was added and to another portion EDTA was added at 0.2%. For the non-EDTA portion the pH was 7.4 and particle size 68 nm. For the portion to which EDTA was added the pH was 7.5 and the particle size 60 nm. For each solution no crystallization was noted following refrigeration of the samples for greater than 560 days.

### Example 33

A composition was made with 0.24% potassium ricinoleate, 0.06% SDS, 0.3% isopropyl alcohol, 0.1% phenoxetol, 0.22% terpineol, 0.04% sodium polyphosphate, and 0.3% PCMX with the remainder deionized water. The solution was mixed gently only. The pH was adjusted with hydrochloric acid (HCl). Crystallization in the solution occurred while mixing and it was noted that the amount of terpineol in the concentrate was under that required. The additional terpineol was added during the RTU (ready-to-use) build. The pH was 7.4 and the particle size was 127 nm for this solution at about 0.3% PCMX. No crystallization was noted following refrigeration of the samples for greater than 554 days.

### Example 34

A composition was made with 0.24% potassium ricinoleate, 0.06% SDS, 0.3% isopropyl alcohol, 0.1% phenoxetol, 0.22% terpineol, 0.01% ricinoleamidopropyl PG-dimonium chloride phosphate, and 0.2% PCMX with the remainder deionized water. The solution was mixed gently only. The pH was adjusted with hydrochloric acid (HCl). To a portion of the mix no EDTA was added and to another portion EDTA was added at 0.1%. For the non-EDTA portion the pH was 7.4 and particle size 86 nm. For the portion to which EDTA was added the pH was 7.3 and the particle size 74 nm. Crystallization in the solution occurred while mixing and it was noted that the amount of terpineol in the concentrate was under that required. The additional terpineol was added during the RTU build. The pH was 7.4 and the particle size was 127 nm for this solution at about 0.3% PCMX. No crystallization was noted following refrigeration of the samples for greater than 549 days.

### Example 35

A composition was made with 0.24% potassium ricinoleate, 0.06% SDS, 0.3% isopropyl alcohol, 0.1% phenoxetol, 0.22% terpineol, 0.04% ricinoleamidopropyl PG-dimonium chloride phosphate, and 0.2% PCMX with the remainder deionized water. The solution was mixed gently only. The pH was adjusted with hydrochloric acid (HCl) and then EDTA added to 0.1%. The pH was 7.3 and the particle size 89 nm. No crystallization was noted following refrigeration of the samples for greater than 547 days.

### Example 36

A composition was made with 0.24% potassium ricinoleate, 0.06% SDS, 0.06% polyoxyethylenesorbitan monopalmitate 40, 0.3% isopropyl alcohol, 0.1% phenoxetol, 0.22% terpineol, and 0.2% PCMX with the remainder deionized water. The solution was mixed gently only. The pH was adjusted with hydrochloric acid (HCl) and then EDTA added to 0.2%. The pH was 7.4 and the particle size 81 nm. No crystallization was noted following refrigeration of the samples for greater than 541 days.

### Example 37

A concentrate was built for the eventual RTU concentrations with 0.24% cetylpyridinium chloride, 0.12% benzyltrimethyl ammonium chloride, 0.04% cocamidopropyl PG-dimonium chloride phosphate, 0.3% isopropyl alcohol, 0.1% phenoxetol, 0.22% terpineol, and 0.2% PCMX. This concentrated emulsion separated out and the RTU was not built.

### Example 38

A composition was made with 0.24% potassium ricinoleate, 1.2% mixture of PEG-8 laurate with laureth-4 and PCMX with resultant concentration of 0.2% PCMX with the remainder deionized water. The solution was mixed gently only. The pH was adjusted with hydrochloric acid (HCl) and then EDTA added to 0.1%. The pH was 7.4 and the particle size 118 nm. No crystallization was noted following refrigeration of the samples for greater than 544 days.

### Example 39

A composition was made with 0.24% potassium ricinoleate, 0.06% SDS, 0.3% isopropyl alcohol, 0.1% phenoxetol, 0.22% terpineol, 0.04% sodium polyphosphate, 1.57% multi-enzyme based solution, and 0.2% PCMX with the remainder deionized water. The solution was mixed gently only. The pH was adjusted with hydrochloric acid (HCl) and EDTA added to 0.2%. The pH was 7.2 and particle size 121 nm. No crystallization was noted in refrigerated samples for greater than 536 days.

### Example 40

A composition was made with 0.24% potassium ricinoleate, 0.06% SDS, 0.35% isopropyl alcohol, 0.05% phenoxetol, 0.22% terpineol, 0.04% sodium polyphosphate, and 0.2% PCMX with the remainder deionized water. The solution was mixed gently only. The pH was adjusted with hydrochloric acid (HCl) and EDTA was added to 0.2%. The pH was 7.4 and particle size 63 nm. No crystallization was noted in refrigerated samples for greater than 810 days. In a second build the pH was 7.4 and the particle size was 62 nm and zeta potential −99 mV. No crystallization was noted in refrigerated samples for greater than 776 days. In a third build the pH was 7.4 and the particle size was 61 nm. No crystallization was noted in refrigerated samples for greater than 771 days. In a fourth build the pH was 7.4, the particle size 66 nm, and the zeta potential −106 mV. No crystallization was noted in refrigerated samples for greater than 741 days. In a fifth build the sodium polyphosphate was 0.24%, pH 7.3, the particle size 66 nm, and the zeta potential −133 mV. No crystallization was noted in refrigerated samples for greater than 706 days. In a sixth build the sodium polyphosphate was 0.25%, the pH 7.3, the particle size 73 nm, and the zeta potential −132 mV. No crystallization was noted in refrigerated samples for greater than 695 days. In a seventh build the sodium polyphosphate was 0.25%, the pH 7.3, the particle size 69 nm, and the zeta potential −119 mV. No crystallization was noted in refrigerated samples for greater than 694 days. In an eighth build the amount of concentrate added to make the RTU was increased by 25% to give a PCMX of about 0.25%. In one portion the pH was adjusted to 8.5 and the particle size was 12 nm and the zeta potential −92 mV. For a second portion the pH was 7.6, the particle size 172 nm, and the zeta potential −106 mV. No crystallization was noted in refrigerated samples for greater than 678 days.

### Example 41

A composition was made with 0.24% potassium ricinoleate, 0.3% isopropyl alcohol, 0.1% phenoxetol, 0.22% terpineol, 0.05% sodium hydroxypropylsulfonate lauryl-glucoside crosspolymer, 0.04% sodium polyphosphate, and 0.2% PCMX with the remainder deionized water. The solution was mixed gently only. The pH was adjusted with hydrochloric acid (HCl) and an additional 0.21% sodium polyphosphate added (total was 0.25%) and 0.2% EDTA added. The pH was 7.4, the particle size 195 nm, and zeta potential −89 mV. No crystallization was noted in refrigerated samples for greater than 804 days.

### Example 42

A composition was made with 0.24% potassium ricinoleate, 0.06% SDS, 0.35% isopropyl alcohol, 0.05% phenoxetol, 0.22% terpineol, 0.04% sodium polyphosphate, and 0.2% PCMX with the remainder deionized water. The solution was mixed gently only. 0.05% tetrasodium 1-hydroxyethylidene-1, 1-diphosphonate (bis-phosphonate) then 0.2% EDTA and then the pH was adjusted with hydrochloric acid (HCl). The pH was 7.7, the particle size 70 nm, and the zeta potential −105 mV. No crystallization was noted in refrigerated samples for greater than 792 days. In a second build the pH was 7.7, the particle size was 79 nm, and zeta potential −98 mV. No crystallization was noted in refrigerated samples for greater than 776 days. In a third build the pH was 7.7, the particle size was 81 nm, and the zeta potential −98 nm. No crystallization was noted in refrigerated samples for greater than 771 days. In a fourth build an additional sodium polyphosphate was added to bring total to 0.125% and bis-phosphonate total was 0.025% the pH was 8.5, the particle size 9 nm, and the zeta potential −100 mV. No crystallization was noted in refrigerated samples for greater than 684 days. In a fifth build the final concentration of EDTA was 0.2% EDTA. Samples were prepared with various pHs of 8.5, 7.4, and 6.9 with particle sizes of 9, 227, and 294 nm respectively; and zeta potentials of −54, −107, and −82 mV. No crystallization was noted in refrigerated samples for greater than 671 days.

In a sixth build the final concentration of sodium xylenesulfonate was 0.2% and the EDTA 0.2%; the pH was 7.6, the particle size 59 nm, and the zeta potential −99 mV. Crystallization was noted in refrigerated samples at 9 days. In a seventh build a portion was made with 0.2% EDTA and the pH was 7.4, the particle size 66 nm, and the zeta potential −103 mV; in a second portion the final EDTA was 0.4% EDTA and the pH 7.5, the particle size 59 nm, and the zeta potential −106 mV. No crystallization was noted in refrigerated samples for greater than 576 days. In an eighth build the final EDTA was 0.2% and the final PCMX was about 0.22%. No crystallization was noted in refrigerated samples for greater than 531. By using a salt test the first built formulation was more stable than the prior art formulation.

### Example 43

A composition was made with 0.24% potassium ricinoleate, 0.06% SDS, 0.35% isopropyl alcohol, 0.05% phenoxetol, 0.22% terpineol, 0.04% sodium polyphosphate, and 0.3% PCMX with the remainder deionized water. The solution was mixed gently only during the build but the crystals appeared during the mixing before all the concentrate was added very likely related to the higher PCMX concentration and the higher PCMX:terpineol ratio.

### Example 44

A composition was made with 0.24% potassium ricinoleate, 0.06% SDS, 0.35% isopropyl alcohol, 0.05% phenoxetol, 0.33% terpineol, 0.04% sodium polyphosphate, and 0.3% PCMX with the remainder deionized water. The solution was mixed gently only. Bis-phosphonate was added to a final concentration of 0.05% and EDTA was added to a final concentration of 0.2% and then the pH was adjusted with hydrochloric acid (HCl). The pH was 7.7, the particle size 187 nm, and the zeta potential −116 mV. At 2 days crystals were seen in the refrigerated sample likely related to the higher PCMX concentration.

### Example 45

A concentrate with a projected RTU composition with about 0.24% potassium ricinoleate, 0.06% SDS, 0.35% isopropyl alcohol, 0.05% phenoxetol, 0.22% terpineol, 0.25% sodium polyphosphate, and 0.2% PCMX was made but the formulation separated before the RTU could be made likely related to the high concentration of the sodium polyphosphate used.

### Example 46

A composition was made with 0.24% potassium ricinoleate, 0.06% SDS, 0.35% isopropyl alcohol, 0.05% phenoxetol, 0.22% terpineol, and 0.2% PCMX with the remainder deionized water. The solution was mixed gently only and sodium polyphosphate (n=21) added at a final concentration of 0.1% and then EDTA added at a final concentration of 0.2% and then the pH adjusted with hydrochloric acid in two portions to 8.1 and 7.4. The particle sizes were 38 nm and 61 nm respectively. Notably in this example and others, the higher the pH the lower the particle size. The zeta potentials were −81 and −99 mV respectively. In a second build made without polyphosphate and an EDTA final concentration of 0.2% and the pH adjusted with hydrochloric acid to a pH of 7.4, the particle size was 62 nm and the zeta potential −93 mV. A third build was made with no polyphosphate but with 0.03% bis-phosphonate and the pH adjusted with hydrochloric acid to 7.4. The particle size was 193 nm and the zeta potential −112 mV. For these formulations no crystallization was noted in refrigerated samples for greater than 598 days.

### Example 47

### Prior Art Formulation but at Higher Concentration

A composition was made using the concentrate formulation as that used for the prior art product but at 1.9 times the concentrate volume typically used. The final composition was about 0.46% potassium ricinoleate, 0.76% isopropyl alcohol, 0.76% terpineol, and 0.38% PCMX with the remainder deionized water. The solution was mixed gently only and then the pH adjusted with hydrochloric acid to 8.5. The particle size was 275 nm and the zeta potential −1.69 mV. While a higher concentration of the concentrate was used this formulation showed a high particle size in the absence of homogenization and a low zeta potential. For this formulation no crystallization was noted in refrigerated samples for greater than 644 days.

### Example 48

A composition was made with 0.18% potassium ricinoleate, 0.06% SDS, 0.35% isopropyl alcohol, 0.05% phenoxetol, 0.22% terpineol, and 0.2% PCMX with the remainder deionized water. The solution was mixed gently only and sodium polyphosphate (n=13) added at a final concentration of 0.04% and then EDTA added at a final concentration of 0.2% and then the pH adjusted with hydrochloric acid to 7.7. The particle size was 50 nm and the zeta potential −88 mV. For this formulation crystals were noted in refrigerated samples at 9 days. This crystallization was believed related to the lower potassium ricinoleate used in the formulation.

### Example 49

A composition was formulated with 0.24% potassium ricinoleate, 1.16% polyoxyethylenesorbitan monopalmitate 40, and 0.2% PCMX with the remainder deionized water. The solution was mixed gently only and sodium polyphosphate (n=13) added at a final concentration of 0.04% and then EDTA added at a final concentration of 0.2% and then the pH adjusted with citric acid to 7.3. The particle size was 113 nm and the zeta potential −32 mV. For this formulation crystals were not noted in refrigerated samples at 623 days.

### Example 50

A composition with the intended concentration of 0.24% potassium ricinoleate, 0.06% SDS, 0.53% isopropyl alcohol, 0.08% phenoxetol, 0.22% terpineol, and 0.3% PCMX with the remainder deionized water. With the addition of the concentrate crystals were formed and the build aborted. The reason for the crystallization is likely related to the high PCMX:terpineol ratio.

### Example 51

A composition was formulated by adding directly to the water a blend of sodium laureth sulfate, D-glucopyranoside, C-6-12-alkyl, oligome, ethanol, and PCMX with the expected final concentration of PCMX of 0.2%. During the build a precipitate was formed around the pH electrode and the run was aborted.

### Example 52

A composition was made with 0.24% potassium ricinoleate, 0.06% SDS, 0.35% isopropyl alcohol, 0.05% phenoxetol, 0.22% terpineol, 0.074% essential oil tea tree heart, and 0.2% PCMX with the remainder deionized water. The solution was mixed gently only and then the pH adjusted with hydrochloric acid to 8.5. The particle size was 73 nm and the zeta potential was −104 mV. No crystallization was noted in refrigerated samples for greater than 595 days.

### Example 53

### Prior Art Formulation but at Higher Concentration

A composition was made using the concentrate formulation as that used for the prior art product but at 1.7 times the concentrate volume typically used. The final composition was about 0.41% potassium ricinoleate, 0.68% isopropyl alcohol, 0.68% terpineol, and 0.34% PCMX with the remainder deionized water. The solution was mixed gently only and then the pH adjusted with hydrochloric acid to 8.5. The particle size was 281 nm and the zeta potential −3.46 mV. While a higher concentration of the concentrate was used this formulation showed a high particle size in the absence of homogenization and a low zeta potential. For this formulation no crystallization was noted in refrigerated samples for greater than 589 days.

### Example 54

A composition was made with 0.24% potassium ricinoleate, 0.06% SDS, 0.35% isopropyl alcohol, 0.05% phenoxetol, 0.22% of the essential oil tea tree heart, 0.04% sodium polyphosphate, and 0.2% PCMX with the remainder deionized water. The solution was mixed gently only and then EDTA added. Then crystals were noted in the solution. Then terpineol was added to a final concentration of 0.22% and then adjusted the pH with hydrochloric acid to a final pH of 7.8. The particle size was 80 nm and the zeta potential −93 mV. For this formulation no crystallization was noted in refrigerated samples for greater than 581 days. In a second build the terpineol was added directly to the concentrate before adding to the water to a final concentration in the RTU of 0.11%. The pH was adjusted with hydrochloric acid to a pH of 7.7. The particle size was 67 nm and the zeta potential −90 mV. For this formulation no crystallization was noted in refrigerated samples for greater than 581 days. In a third build the RTU was pH adjusted with hydrochloric acid and then EDTA added to a final concentration of 0.2%. The pH was 7.6, the particle size 52 nm, and the zeta potential −54 mV. For this formulation no crystallization was noted in refrigerated samples for greater than 577 days.

### Example 55

### Prior Art Formulation but at Higher Concentration

A composition was made using the concentrate formulation as that used for the prior art product but at 1.75 times the concentrate volume typically used. The final composition was 0.42% potassium ricinoleate, 0.7% isopropyl alcohol, 0.7% terpineol, and 0.35% PCMX with the remainder deionized water. The solution was mixed gently only and then the pH adjusted with hydrochloric acid to 8.5. The particle size was 276 nm and the zeta potential −2.96 mV. While a higher concentration of the concentrate was used, this formulation showed a high particle size in the absence of homogenization and a low zeta potential. For this formulation no crystallization was noted in refrigerated samples for greater than 575 days.

### Example 56

A composition was made with 0.24% potassium ricinoleate, 0.06% SDS, 0.35% isopropyl alcohol, 0.05% phenoxetol, 0.22% terpineol, 0.04% sodium polyphosphate, and 0.2% PCMX with the remainder deionized water. The solution was mixed gently only and the pH was adjusted with citric acid and EDTA added to a final concentration of 0.3%. The pH was 7.9, the particle size 39 nm, and the zeta potential −84 mV. For this formulation no crystallization was noted in refrigerated samples for greater than 559 days. In a second build the pH was adjusted with citric acid and EDTA added to a final concentration of 0.4%. The pH was 7.9, the particle size was 62 nm, and the zeta potential −107 mV. For this formulation no crystallization was noted in refrigerated samples for greater than 556 days. In a third build the pH was adjusted with citric acid and EDTA added to a final concentration of 0.5%. The pH was 7.9, the particle size was 25 nm, and the zeta potential −120 mV. For this formulation no crystallization was noted in refrigerated samples for greater than 555 days.

### Example 57

A composition was made with 0.24% potassium ricinoleate, 0.03% SDS, 0.35% isopropyl alcohol, 0.05% phenoxetol, 0.22% terpineol, 0.04% sodium polyphosphate, and 0.2% PCMX with the remainder deionized water. The solution was mixed gently only and the pH was adjusted with hydrochloric acid and EDTA added to a final concentration of 0.2%. The pH was 8.0, the particle size 62 nm, and the zeta potential −100 mV. For this formulation no crystallization was noted in refrigerated samples for greater than 547 days. In a second build the pH was adjusted with hydrochloric acid and EDTA added to a final concentration of 0.2% and trisodium nitrilotriacetic acid added to a final concentration of 0.1%. The pH was 8.49, the particle size was 29 nm, and the zeta potential −101 mV. For this formulation no crystallization was noted in refrigerated samples for greater than 542 days where the sample was in a glass bottle and crystals were seen at 546 days when the sample was stored in HDPE.

### Example 58

A concentrate was built with an RTU of 0.24% SDS, 0.35% isopropyl alcohol, 0.05% phenoxetol, 0.22% terpineol, 0.04% sodium polyphosphate, and 0.2% PCMX. During the build when the polyphosphate was added the pH dropped below 7 and it was adjusted upward with concentrated potassium hydroxide. In time this formulation showed separation and in a trial RTU a precipitate formed and the trial aborted. This was likely related to the absence of potassium ricinoleate in the formulation.

### Example 59

A composition was made with 0.24% potassium ricinoleate, 0.06% SDS, 0.35% isopropyl alcohol, 0.05% phenoxetol, 0.22% terpineol, 0.04% sodium polyphosphate, and 0.2% mixed phenols (0.05% PCMX, 0.05% 2-benzl-4-chlorophenol, 0.05% para tertiary amylphenol, and 0.05% ortho-phenyl-phenol) with the remainder deionized water. The solution was mixed gently only and the pH was adjusted with hydrochloric acid and EDTA added to a final concentration of 0.2%. The pH was 7.9, the particle size 52 nm, and the zeta potential −85 mV. For this formulation no crystallization was noted in refrigerated samples for greater than 535 days.

### Example 60

### Prior Art Formulation but at Higher Concentration

A composition was made using the concentrate formulation as that used for the prior art product but at 1.6 times the concentrate volume typically used. The final composition was 0.38% potassium ricinoleate, 0.64% isopropyl alcohol, 0.64% terpineol, and 0.32% PCMX with the remainder deionized water. The solution was mixed gently only and then the pH adjusted with hydrochloric acid to 8.5. The particle size was 261 nm and the zeta potential −4.56 mV. While a higher concentration of the concentrate was used, this formulation showed a high particle size in the absence of homogenization and a low zeta potential.

### Example 61

### Prior Art Formulation but at Higher Concentration

A composition was made using the same concentrate formulation as that used for the prior art product but at 1.725 times the concentrate volume typically used. The final composition was 0.41% potassium ricinoleate, 0.69% isopropyl alcohol, 0.69% terpineol, and 0.345% PCMX with the remainder deionized water. The solution was mixed gently only and then the pH adjusted with hydrochloric acid to 8.5. The particle size was 274 nm and the zeta potential −3.01 mV. While a higher concentration of the concentrate was used, this formulation showed a high particle size in the absence of homogenization and a low zeta potential.

### Example 62

A composition was made with 0.24% potassium ricinoleate, 0.06% SDS, 0.35% isopropyl alcohol, 0.05% phenoxetol, 0.22% terpineol, 0.04% sodium polyphosphate, and 0.22% PCMX with the remainder deionized water. The solution was mixed gently only and the pH was adjusted with hydrochloric acid and EDTA added to a final concentration of 0.2%. The pH was 7.4, the particle size 59 nm, and the zeta potential −101 mV. For this formulation no crystallization was noted in refrigerated samples for greater than 503 days. In a second build made similarly, the pH was 7.4, the particle size 60 nm, and the zeta potential −103 mV. For this formulation no crystallization was noted in refrigerated samples for greater than 500 days.

### Example 63

A composition was made with 0.012% potassium ricinoleate, 0.02% isopropyl alcohol, 0.02% terpineol, and 0.01% PCMX with the remainder deionized water. The solution was mixed gently only and the pH was not adjusted. The pH was 7.3, the particle size 22 nm, and the zeta potential −0.623 mV. For this formulation no crystallization was noted in refrigerated samples for greater than 374 days.

### Example 64

A composition was made with 0.24% potassium ricinoleate, 0.06% SDS, 0.35% isopropyl alcohol, 0.05% phenoxetol, 0.22% terpineol, 0.04% sodium polyphosphate, and 0.22% PCMX with the remainder deionized water. The solution was mixed gently only and the pH was adjusted with hydrochloric acid and EDTA added to a final concentration of 0.2%. The pH was 7.4, the particle size 56 nm, and the zeta potential −98 mV. For this formulation crystallization was noted in refrigerated samples at 224 days. In a second build the PCXM concentration was about 0.353% and the other components increased proportionally. The pH was 7.9, the particle size 51 nm, and the zeta potential −100 mV. For this formulation crystallization was not noted in refrigerated samples at 465 days.

### Example 65

A composition was made with 0.24% potassium ricinoleate, 0.06% SDS, 0.35% isopropyl alcohol, 0.05% phenoxetol, 0.26% terpineol, 0.04% sodium polyphosphate, and 0.22% PCMX with the remainder deionized water. The solution was mixed gently only and the pH was adjusted with hydrochloric acid and EDTA added to a final concentration of 0.2%. The pH was 7.5, the particle size 60 nm, and the zeta potential −95 mV. For this formulation crystallization was noted in refrigerated samples at 478 days. In a second build the PCXM concentration was about 0.353% and the other components increased proportionally. The pH was adjusted with hydrochloric acid and EDTA added to a final concentration of 0.2%. The pH was 7.9, the particle size 55 nm, and the zeta potential −102 mV. For this formulation no crystallization was noted in refrigerated samples for greater than 464 days. In a third build the PCXM concentration was about 0.27% and the other components increased proportionally. The pH was adjusted with hydrochloric acid and EDTA added to a final concentration of 0.2%. The pH was 7.9, the particle size 56 nm, and the zeta potential −95 mV. For this formulation no crystallization was noted in refrigerated samples for greater than 448 days. In a fourth build (like the third build) the PCXM concentration was 0.27% and the other components increased proportionally. The pH was adjusted with hydrochloric acid and EDTA added to a final concentration of 0.2%. The pH was 7.9, the particle size 53 nm, and the zeta potential −95 mV. For this formulation no crystallization was noted in refrigerated samples for greater than 435 days. In a fifth build the PCXM concentration was 0.30% and the other components increased proportionally. The pH was adjusted with hydrochloric acid and EDTA added to a final concentration of 0.2%. The pH was 7.9, the particle size 59 nm, and the zeta potential −99 mV. For this formulation no crystallization was noted in refrigerated samples for greater than 426 days. In a sixth build (like fifth build) the PCXM concentration was 0.30% and the other components increased proportionally. The pH was adjusted with hydrochloric acid and EDTA added to a final concentration of 0.2%. The pH was 7.9, the particle size 59 nm, and the zeta potential −100 mV.

### Example 66

A composition was made with 0.24% potassium ricinoleate, 0.06% SDS, 0.47% isopropyl alcohol, 0.07% phenoxetol, 0.29% terpineol, 0.04% sodium polyphosphate, and 0.27% PCMX with the remainder deionized water. The solution was mixed gently only and the pH was adjusted with hydrochloric acid and EDTA added to a final concentration of 0.2%. The pH was 7.9, the particle size 58 nm, and the zeta potential −99 mV. For this formulation crystallization was noted in refrigerated samples at 9 days.

### Example 67

A composition was made with 0.36% potassium ricinoleate, 0.09% SDS, 0.53% isopropyl alcohol, 0.075% phenoxetol, 0.45% terpineol, 0.06% sodium polyphosphate, and 0.3% PCMX with the remainder deionized water. The solution was mixed gently only and the pH was adjusted with hydrochloric acid and EDTA added to a final concentration of 0.2%. The pH was 7.9, the particle size 62 nm, and the zeta potential −100 mV. For this formulation crystallization was not noted in refrigerated samples at 420 days. In a second build the composition made was 0.32% potassium ricinoleate, 0.08% SDS, 0.47% isopropyl alcohol, 0.07% phenoxetol, 0.41% terpineol, 0.05% sodium polyphosphate, and 0.27% PCMX with the remainder deionized water. The solution was mixed gently only and the pH was adjusted with hydrochloric acid and EDTA added to a final concentration of 0.2%. The pH was 7.9, the particle size 61 nm, and the zeta potential −101 mV. For this formulation crystallization was not noted in refrigerated samples at 380 days.

### Example 68

A composition was made with 0.01% potassium ricinoleate, 0.02% propylene glycol, 0.02% terpineol, and 0.01% PCMX with the remainder deionized water. The solution was mixed gently only and the pH was not adjusted. The pH was 7.5, the particle size 23 nm, and the zeta potential −86 mV. For this formulation crystallization was not noted in refrigerated samples at 374 days.

### Example 69

A composition was made with 0.3% potassium ricinoleate, 0.075% SDS, 0.44% isopropyl alcohol, 0.06% phenoxetol, 0.273% terpineol, 0.05% sodium polyphosphate, and 0.25% PCMX with the remainder deionized water. The solution was mixed gently only and the pH was adjusted with hydrochloric acid and EDTA added to a final concentration of 0.2%. The pH was 7.8, the particle size 80 nm, and the zeta potential −108 mV. For this formulation crystallization was noted in refrigerated samples at 289 days. A second composition was formulated with similar chemistries except to the solution was added 0.1% 3-(trihydroxysilyl) propyldimethyloctadecyl ammonium chloride. The pH was adjusted with hydrochloric acid and EDTA added to a final concentration of 0.2%. The pH was 7.6, the particle size 769 nm, and the zeta potential −35 mV. For this formulation no crystallization was noted in refrigerated samples at 274 days. A third composition was formulated with similar chemistries (and like the second formulation) and 0.1% 3-(trihydroxysilyl) propyldimethyloctadecyl ammonium chloride was added. The pH was adjusted with hydrochloric acid and EDTA added to a final concentration of 0.2%. The pH was 7.8, the particle size 565 nm, and the zeta potential −35 mV. For this formulation no crystallization was noted in refrigerated samples at 273 days. A fourth composition was formulated with 0.3% potassium ricinoleate, 0.075% SDS, 0.44% isopropyl alcohol, 0.06% phenoxetol, 0.273% terpineol, 0.05% sodium polyphosphate, and 0.25% PCMX with the remainder deionized water. The solution was mixed gently only and the pH was adjusted with hydrochloric acid and EDTA added to a final concentration of 0.2%. The pH was 7.8, the particle size 87 nm, and the zeta potential −110 mV. For this formulation crystallization was not noted in refrigerated samples at 176 days.

### Example 70

A composition was made with 0.3% potassium ricinoleate, 0.075% SDS, 0.44% isopropyl alcohol, 0.06% phenoxetol, 0.273% terpineol, 0.05% sodium polyphosphate, and 0.25% PCMX with the remainder deionized water. The solution was mixed gently only and the pH was adjusted with hydrochloric acid and EDTA added to a final concentration of 0.2%. The pH was 7.8, the particle size 80 nm, and the zeta potential −101 mV. For this formulation crystallization was not noted in refrigerated samples at 288 days.

### Example 71

A composition was made with 0.3% potassium ricinoleate, 0.075% SDS, 0.44% isopropyl alcohol, 0.06% phenoxetol, 0.26% terpineol, 0.01% lemongrass essential oil, 0.05% sodium polyphosphate, and 0.25% PCMX with the remainder deionized water. The solution was mixed gently only and the pH was adjusted with hydrochloric acid and EDTA added to a final concentration of 0.2%. The pH was 7.9, the particle size 49 nm, and the zeta potential −99 mV. For this formulation crystallization was not noted in refrigerated samples at 48 days but crystals were seen at 140 days.

### Example 72

A composition (like that of Example 71 except a different manufacturer's lemongrass essential oil was used) with about 0.3% potassium ricinoleate, 0.075% SDS, 0.44% isopropyl alcohol, 0.06% phenoxetol, 0.26% terpineol, 0.01% lemongrass essential oil, 0.05% sodium polyphosphate, and 0.25% PCMX with the remainder deionized water. The solution was mixed gently only and the pH was adjusted with hydrochloric acid and EDTA added to a final concentration of 0.2%. The pH was 7.7, the particle size 48 nm, and the zeta potential −103 mV. For this formulation crystallization was not noted in refrigerated samples at 133 days.

### Example 73

A composition was made with 0.3% potassium ricinoleate, 0.075% SDS, 0.44% isopropyl alcohol, 0.06% phenoxetol, 0.273% terpineol, 0.05% sodium polyphosphate, and 0.25% PCMX with the remainder deionized water. The solution was mixed gently only and the pH was adjusted with hydrochloric acid and EDTA added to a final concentration of 0.2%. The pH was 7.9, the particle size 67 nm, and the zeta potential −96 mV. A second formulation was made with similar properties as that of the first build. The solution was mixed gently only and the pH was adjusted with hydrochloric acid and EDTA added to a final concentration of 0.2%. The pH was 8.0, the particle size 64 nm, and the zeta potential −106 mV. A third formulation was made with similar properties as that of the first and second builds. The solution was mixed gently only and the pH was adjusted with hydrochloric acid and EDTA added to a final concentration of 0.2%. The pH was 7.9, the particle size 71 nm, and the zeta potential −99 mV. In evaluating these solutions over time pHs, particle sizes and zeta potential were stable. In microbiological analyses per the AOAC Use Dilution tests in an independent lab these lots passed for Staphylococcus aureus (ATCC 6538), Pseudomonas aeruginosa (ATCC 15442), and Salmonella enterica (ATCC 10708) with an organic load in at most 10 minutes. In addition, this formulation tested in an independent lab passed the acute eye irritation test and met criteria for Toxicity Category IV. The first formulation was tested in the presence of 5% fetal bovine serum in an independent lab per the AOAC Germicidal Spray Method. At about 10 minutes contact time there were no positives in 60 carriers tested; at about 5 minutes there were two carriers positive in 60 carriers tested. All three formulations were evaluated in an independent laboratory in a GLP study per the AOAC Germicidal Spray Method. The organisms Staphylococcus aureus (ATCC 6538), Pseudomonas aeruginosa (ATCC 15442), and Salmonella enterica (ATCC 10708) were assessed at 9 minutes and 45 seconds at room temperature (20° C.) with 5% fetal bovine serum organic soil. The carrier counts were: Staphylococcus aureus=5.4×10⁶ CFU/carrier, Pseudomonas aeruginosa=4.6×10⁶ CFU/carrier, and Salmonella enterica=4.4×10⁵ CFU/carrier. For lot #1 Staphylococcus aureus=0/60 subculture tubes demonstrated growth of the test organism PASSED), Pseudomonas aeruginosa=0/60 subculture tubes demonstrated growth of the test organism (PASSED), Salmonella enterica=0/60 subculture tubes demonstrated growth of the test organism (PASSED). For the second lot Staphylococcus aureus=0/60 subculture tubes demonstrated growth of the test organism (PASSED), Pseudomonas aeruginosa=0/60 subculture tubes demonstrated growth of the test organism (PASSED), Salmonella enterica=0/60 subculture tubes demonstrated growth of the test organism (PASSED). For the third lot Staphylococcus aureus=0/60 subculture tubes demonstrated growth of the test organism (PASSED), Pseudomonas aeruginosa=0/60 subculture tubes demonstrated growth of the test organism (PASSED), Salmonella enterica=0/60 subculture tubes demonstrated growth of the test organism (PASSED). Two lots of this formulation were evaluated in an independent laboratory per US EPA approved methods by a germicidal spray test for a virucide for Influenza A virus (ATCC VR-544, Strain Hong Kong at 5 minutes, room temperature (20° C.) with 5% fetal bovine serum organic soil. These two lots evaluated against carriers with carrier counts of 6.75×10⁶ CFU/carrier showed PASSED with greater than or equal to 6.25 log reductions. Two lots of this formulation were evaluated in an independent laboratory per US EPA approved fungicidal germicidal spray for Candida albicans (ATCC 10231) at 9 minutes and 45 seconds at room temperature (23.5° C.) with 5% fetal bovine serum organic soil. These lots evaluated against carriers with carrier counts of 0.62×10⁵ CFU/carrier showed PASSED with demonstrated efficacy. Two lots of this formulation were evaluated in an independent laboratory per US EPA approved methods by a germicidal spray test for a virucide for Avian Influenza A virus (H3N2, ATCC VR-2072, Strain A/Washington/897/80xA/Mallard/New york/6750/78) at 5 minutes, room temperature (20° C.) with 5% fetal bovine serum organic soil. These two lots evaluated against carriers with carrier counts of 5.5 logs showed PASSED with complete inactivation with greater than or equal to 4.5 log reductions. Two lots of this formulation were evaluated in an independent laboratory per US EPA approved methods by a germicidal spray test for a virucide for Herpes simplex virus Type 1, ATCC VR-733, Strain F(1) at 5 minutes, room temperature (20° C.) with 5% fetal bovine serum organic soil. These two lots evaluated against carriers with carrier counts of 5.5 logs showed PASSED with complete inactivation with greater than or equal to 5.0 log reductions. Two lots of this formulation were evaluated in an independent laboratory per US EPA approved methods by a germicidal spray test for a virucide for Herpes simplex virus Type 2, ATCC VR-734, Strain G at 5 minutes, room temperature (20° C.) with 5% fetal bovine serum organic soil. These two lots evaluated against carriers with carrier counts of 4.75 logs showed PASSED with complete inactivation with greater than or equal to 4.25 log reductions.

Additional testing was carried out on formulation lots 2 and 3 at a PCMX concentration of 0.18% each for the “Big 3”. These formulations which were over 2 years old were evaluated in an independent laboratory in a GLP study per the AOAC Germicidal Spray Method. The organisms Staphylococcus aureus (ATCC 6538), Pseudomonas aeruginosa (ATCC 15442), and Salmonella enterica (ATCC 10708) were assessed at 9 minutes and 45 seconds at room temperature (21-22° C.) with 5% fetal bovine serum organic soil. The carrier counts were: Staphylococcus aureus=3.16×10⁶ CFU/carrier (6.50 logs), Pseudomonas aeruginosa=1.15×10⁶ CFU/carrier (6.04 logs), and Salmonella enterica=1.23×104 CFU/carrier (4.41 logs). For lot #2 Staphylococcus aureus=0/60 subculture tubes demonstrated growth of the test organism PASSED), Pseudomonas aeruginosa=0/60 subculture tubes demonstrated growth of the test organism (PASSED), Salmonella enterica=0/60 subculture tubes demonstrated growth of the test organism (PASSED). For lot #3 Staphylococcus aureus=0/60 subculture tubes demonstrated growth of the test organism (PASSED), Pseudomonas aeruginosa=0/60 subculture tubes demonstrated growth of the test organism (PASSED), Salmonella enterica=0/60 subculture tubes demonstrated growth of the test organism (PASSED).

### Example 74

### 03242011, T70

A concentrate composition was made with 28.3% sodium laureth sulfate, 9.5% decyl glucoside, 10.24% PCMX, 30.0% propylene glycol with no added deionized water. Without pH adjustment the pH was about 8.5-9.

Prepared a Ready-to-Use solution by adding 2.5 ml of the concentrate to 100 ml of deionized water (about a 2500 ppm PCMX concentration) the solution was crystal clear with no ppt., nor oil. Refrigerated through 101 days solution remained crystal clear.

### Example 75

### 110910, MC1084

A concentrate composition was made with 30.0% sodium laureth sulfate, 10% glucoside, 10.25% PCMX, 10.9% terpineol, 15.7% propylene glycol with no added deionized water. Without pH adjustment the pH was about 10. With pH adjustment with concentrated HCl the pH was 8.55

In an independent laboratory, microbiological testing was conducted with the formulation at about 200 ppm PCMX in deionized water and 2.5% sodium chloride solutions at room temperature (23±2° C.). The organisms studied were Bacillus cereus ATCC#14579, Pseudomonas fluorescens ATCC#13525, and Aspergillus niger spores ATCC#9642. The contact times for testing were less than one minute, 1 and 3 hours. For the vegetative bacteria Bacillus cerus and Pseudomonas fluorescens at all times and for both organisms the number of organisms remaining were <1 organism (no growth). The log reductions were greater than 4 logs demonstrating significant antibacterial activity. For the A. niger spores tested with deionized water and 2.5% sodium chloride the reductions were 58.75% and 77.78% at 3 hours respectively.

Prepared a Ready-to-Use solution by adding 2.5 ml of the concentrate (MC 1084) to 100 ml of deionized water (about a 2500 ppm PCMX concentration) the solution was crystal clear with no ppt., nor oil. Refrigerated through 101 days solution remained crystal clear.

### Example 76

A composition was formulated with 7.3% sodium laureth sulfate, 1.4% lauryl glucoside (C10-16), lauryl glucoside (C6-12), 1.2% cocamidopropyl betaine, 0.15% ethanol, 0.26% PCMX, 0.8% sodium chloride, 2.5% cocamidopropyl PG-dimonium chloride phosphate, 0.08% 1,3-dihydroxymethyl-5,5-dimethylhydantoin with the remainder water. The final pH was 6.87.

### Example 77

A composition was formulated with 7.3% sodium laureth sulfate, 1.4% lauryl glucoside (C10-16), lauryl glucoside (C6-12), 1.2% cocamidopropyl betaine, 0.15% ethanol, 0.25% PCMX, 1.55% sodium chloride, 2.5% cocamidopropyl PG-dimonium chloride phosphate, 6.9% hydrogenated starch hydrolysate, 0.5% polyquaternium-10, 0.2% ethylenediamine-tetraacetic acid trisodium salt hydrate, and 0.002% lemon fragrance with the remainder water. The final pH was 6.8.

Microbiological assays were conducted on this formulation through an independent laboratory. The sample was evaluated for microbial content and found to be free from contamination. In a preservation test 0.11% 1,3-dihydroxymethyl-5,5-dimethylhydantoin was shown to adequately preserve the formulation.

### Example 78

A composition was formulated with 7.3% sodium laureth sulfate, 1.4% lauryl glucoside (C10-16), lauryl glucoside (C6-12), 1.2% cocamidopropyl betaine, 0.15% ethanol, 0.25% PCMX, 0.8% sodium chloride, 2.5% cocamidopropyl PG-dimonium chloride phosphate, 4% lauryl glucoside hydroxypropyl sulphonate, 6.9% hydrogenated starch hydrolysate, 0.5% polyquaternium-10, and 0.2% ethylenediaminetetraacetic acid trisodium salt hydrate with the remainder water. The final pH was 6.3.

### Example 79

A composition was formulated with 7.3% sodium laureth sulfate, 1.4% lauryl glucoside (C10-16), lauryl glucoside (C6-12), 1.2% cocamidopropyl betaine, 0.15% ethanol, 0.25% PCMX, 0.88% sodium chloride, 2.5% cocamidopropyl PG-dimonium chloride phosphate, 6.9% hydrogenated starch hydrolysate, 0.25% polyquaternium-10, and 0.2% ethylenediaminetetraacetic acid trisodium salt hydrate, and 0.49% honey lemon or 0.74% citrus mint with the remainder water. The final pH was 7.0. The viscosity of the formulation without added fragrance at 24 C was 189 Zahn seconds and 2144 Zahn seconds with the citrus mint fragrance.

### Example 80

A composition was formulated with 7.3% sodium laureth sulfate, 1.4% lauryl glucoside (C10-16), lauryl glucoside (C6-12), 1.2% cocamidopropyl betaine, 0.18% ethanol, 0.25% PCMX, 0.8% sodium chloride, 2.5% cocamidopropyl PG-dimonium chloride phosphate, 6.9% hydrogenated starch hydrolysate, 0.25% polyquaternium-10, and 0.2% ethylenediaminetetraacetic acid trisodium salt hydrate with the remainder water. The final pH was 7.0. The viscosity of the formulation at 23 C without added fragrance was 105 Zahn seconds with a G3 viscometer and 43 Zahn seconds with a G5 viscometer. With 0.25% tea tree peppermint fragrance the viscosity at 23 C was 582 Zahn seconds with a G3 viscometer and 289 Zahn seconds with a G5 viscometer. With 0.26% lemon fragrance the viscosity at 23 C was 386 Zahn seconds with a G3 viscometer and 146 Zahn seconds with a G5 viscometer.

### Example 81

A composition was formulated with 7.4% sodium laureth sulfate, 1.4% lauryl glucoside (C10-16), lauryl glucoside (C6-12), 1.2% cocamidopropyl betaine, 0.19% ethanol, 0.31% PCMX, 0.8% sodium chloride, 2.5% cocamidopropyl PG-dimonium chloride phosphate, 0.2% ethylenediaminetetraacetic acid trisodium salt hydrate, 0.11% 1,3-dihydroxymethyl-5,5-dimethylhydantoin, 0.35% fragrance oil, peppermint Yakima red, tea tree with the remainder water. The final pH was 6.96.

### Example 82

A composition was formulated with 7.4% sodium laureth sulfate, 1.4% lauryl glucoside (C10-16), lauryl glucoside (C6-12), 1.2% cocamidopropyl betaine, 0.19% ethanol, 0.31% PCMX, 0.8% sodium chloride, 2.5% cocamidopropyl PG-dimonium chloride phosphate, 0.2% ethylenediaminetetraacetic acid trisodium salt hydrate, 0.11% 1,3-dihydroxymethyl-5,5-dimethylhydantoin, 0.50% fragrance oil, peppermint Yakima red, tea tree with the remainder water. The final pH was 6.95.

### Example 83

A composition was formulated with 7.4% sodium laureth sulfate, 1.4% lauryl glucoside (C10-16), lauryl glucoside (C6-12), 1.2% cocamidopropyl betaine, 0.19% ethanol, 0.31% PCMX, 0.8% sodium chloride, 2.5% cocamidopropyl PG-dimonium chloride phosphate, 0.2% ethylenediaminetetraacetic acid trisodium salt hydrate, 0.11% 1,3-dihydroxymethyl-5,5-dimethylhydantoin, 0.75% fragrance oil, peppermint Yakima red, tea tree with the remainder water. The final pH was 6.76. The viscosity of the formulation was 2363 Zahn seconds at 22 C with a G5 viscometer.

Microbiological preservation assays were conducted on this formulation through an independent laboratory. The sample was evaluated to be free from contamination and adequately preserved as shown by its ability to withstand numerous inoculations. Time kill conducted on the samples showed Escherichia coli ATCC 8739 (baseline count of 1,766,000) was reduced 92.690% in 60 seconds and 95.367% in 120 seconds; Staphylococcus aureus ATCC 6538 (baseline count of 4,455,000) was reduced 97.817% in 60 seconds and 98.469% in 120 seconds; Pseudomonas aeruginosa ATCC 15442 (baseline count of 5,091,000) was reduced 99.992% in 60 seconds and 99.997% in 120 seconds; and Methicillin Resistant Staphylococcus aureus ATCC 33691 (MRSA) (baseline count of 1,351,000) was reduced 95.855% in 60 seconds and 98.609% in 120 seconds.

### Example 84

A composition was formulated with 2.7% sodium laureth sulfate, 0.6% lauryl glucoside (C10-16), lauryl glucoside (C6-12), 0.4% cocamidopropyl betaine, 0.19% ethanol, 0.31% PCMX, 0.8% sodium chloride, 1.25% cocamidopropyl PG-dimonium chloride phosphate, 0.41% hydrogenated starch hydrolysate, 0.6% glycerin, 0.2% ethylenediaminetetraacetic acid trisodium salt hydrate, 0.11% 1,3-dihydroxymethyl-5,5-dimethylhydantoin, 0.06% allantoin without fragrance and with citric acid for pH adjustment and with the remainder water. The final pH was 5.99. This base formulation was mixed with fragrance of 0.16% oil of cucumber aloe. Also this base formulation was mixed with fragrance of 0.64% water soluble cucumber aloe. This formulation was packaged in a foaming dispenser and dispensed foam.

### Example 85

A composition was formulated with 2.7% sodium laureth sulfate, 0.6% lauryl glucoside (C10-16), lauryl glucoside (C6-12), 0.4% cocamidopropyl betaine, 0.19% ethanol, 0.32% PCMX, 0.8% sodium chloride, 1.25% cocamidopropyl PG-dimonium chloride phosphate, 0.41% hydrogenated starch hydrolysate, 0.6% glycerin, 0.2% ethylenediaminetetraacetic acid trisodium salt hydrate, 0.11% 1,3-dihydroxymethyl-5,5-dimethylhydantoin, 0.06% allantoin with 0.5% fragrance water soluble cucumber aloe and with citric acid for pH adjustment and with the remainder water. The final pH was 5.48. This formulation was packaged in a foaming dispenser and dispensed foam.

Microbiological preservation assays were conducted on this formulation through an independent laboratory. The sample was evaluated to be free from contamination and adequately preserved. Time kill conducted on the samples showed Escherichia coli ATCC 8739 (baseline count of 7,459,000) was reduced 99.589% in 60 seconds and 99.762% in 120 seconds; Staphylococcus aureus ATCC 6538 (baseline count of 3,636,000) was reduced 98.294% in 60 seconds and 99.099% in 120 seconds; Pseudomonas aeruginosa ATCC 15442 (baseline count of 7,454,000) was reduced 99.109% in 60 seconds and 99.134% in 120 seconds; and Methicillin Resistant Staphylococcus aureus ATCC 33691 (MRSA) (baseline count of 1,711,000) was reduced 99.027% in 60 seconds and 99.819% in 120 seconds.

### Example 86

A composition was formulated with 7.4% sodium laureth sulfate, 1.4% lauryl glucoside (C10-16), lauryl glucoside (C6-12), 1.2% cocamidopropyl betaine, 0.19% ethanol, 0.32% PCMX, 0.8% sodium chloride, 2.50% cocamidopropyl PG-dimonium chloride phosphate, 0.2% ethylenediaminetetraacetic acid trisodium salt hydrate, 0.11% 1,3-dihydroxymethyl-5,5-dimethylhydantoin with 0.75% fragrance oil peppermint Yakima redistilled tea tree and with the remainder water. The final pH was 6.79. The viscosity at 22 C was 1898 Zahn seconds with a G5 viscometer. As measured in a standard viscometer the viscosity was 7,100 cps. This formulation when appropriately diluted was dispensable as a foam.

### Example 87

A composition was formulated with 7.4% sodium laureth sulfate, 1.4% lauryl glucoside (C10-16), lauryl glucoside (C6-12), 1.2% cocamidopropyl betaine, 0.19% ethanol, 0.32% PCMX, 0.8% sodium chloride, 2.50% cocamidopropyl PG-dimonium chloride phosphate, 0.2% ethylenediaminetetraacetic acid trisodium salt hydrate, 0.11% 1,3-dihydroxymethyl-5,5-dimethylhydantoin with 0.75% fragrance oil mint tea tree heart and with the remainder water. The final pH was 6.92. The viscosity at 23 C was 1042 Zahn seconds with a G5 viscometer (sample contained many bubbles). Repeat testing at 24 C the Zahn viscosity was 424 Zahn seconds. As measured in a standard viscometer the viscosity was >10,000 cps.

### Example 88

A composition was formulated with 7.4% sodium laureth sulfate, 1.4% lauryl glucoside (C10-16), lauryl glucoside (C6-12), 1.2% cocamidopropyl betaine, 0.19% ethanol, 0.32% PCMX, 0.8% sodium chloride, 0.20% cocamidopropyl PG-dimonium chloride phosphate, 0.2% ethylenediaminetetraacetic acid trisodium salt hydrate, 0.11% 1,3-dihydroxymethyl-5,5-dimethylhydantoin with 0.75% fragrance oil mint tea tree heart and with the remainder water. The formulation had a pH of about 6-6.5 as measured with pH strips. The viscosity at 24 C was 9 Zahn seconds with a G3 viscometer. For the formulation with 0.05% sodium chloride the viscosity was 9.3 Zahn seconds with a G3 viscometer. For the formulation with 0.075% sodium chloride the viscosity was 9.7 Zahn seconds. For the formulation with 0.15% sodium chloride the viscosity was 11 Zahn seconds. For the formulation with 0.30% sodium chloride the viscosity was 21.7 Zahn seconds. For the formulation with 0.60% sodium chloride the viscosity was 168 Zahn seconds. For the formulation with 1.00% sodium chloride the viscosity was 539 Zahn seconds. For the formulation with 0.50% sodium chloride the viscosity was 81 Zahn seconds. For the formulation with 0.515% sodium chloride the viscosity was 104 Zahn seconds. For the formulation with 0.52% sodium chloride the viscosity was 137 Zahn seconds.

### Example 89

A composition was formulated with 7.4% sodium laureth sulfate, 1.4% lauryl glucoside (C10-16), lauryl glucoside (C6-12), 1.2% cocamidopropyl betaine, 0.19% ethanol, 0.32% PCMX, 0.8% sodium chloride, 0.20% cocamidopropyl PG-dimonium chloride phosphate, 0.2% ethylenediaminetetraacetic acid trisodium salt hydrate, 0.11% 1,3-dihydroxymethyl-5,5-dimethylhydantoin with 0.75% fragrance oil mint tea tree heart, 0.50% sodium chloride and with the remainder water. The formulation had a pH of 6.61. The viscosity at 25 C was 70.3 Zahn seconds with a G3 viscometer. The viscosity measured with a standard viscometer was 1,500 cps.

### Example 90

A composition similar to that of Example 89 was formulated with 7.4% sodium laureth sulfate, 1.4% lauryl glucoside (C10-16), lauryl glucoside (C6-12), 1.2% cocamidopropyl betaine, 0.19% ethanol, 0.32% PCMX, 0.8% sodium chloride, 0.20% cocamidopropyl PG-dimonium chloride phosphate, 0.2% ethylenediaminetetraacetic acid trisodium salt hydrate, 0.11% 1,3-dihydroxymethyl-5,5-dimethylhydantoin with 0.75% fragrance oil mint tea tree heart, 0.50% sodium chloride and with the remainder water. The formulation had a pH of 6.60. The viscosity at 24.5 C was 64 Zahn seconds with a G3 viscometer. The viscosity measured with a standard viscometer was 1,370 cps.

Microbiological preservation assays were conducted on this formulation through an independent laboratory. The sample was evaluated to be free from contamination and adequately preserved. Time kill conducted on the samples showed Escherichia coli ATCC 8739 (baseline count of 1,505,000) was reduced 95.832% in 60 seconds and 96.744% in 120 seconds; Staphylococcus aureus ATCC 6538 (baseline count of 1,315,000) was reduced 98.294% in 60 seconds and 94.608% in 120 seconds; Pseudomonas aeruginosa ATCC 15442 (baseline count of 718,200) was reduced 92.219% in 60 seconds and 97.671% in 120 seconds; and Methicillin Resistant Staphylococcus aureus ATCC 33691 (MRSA) (baseline count of 1,711,000) was reduced 94.102% in 60 seconds and 94.209% in 120 seconds.

This formulation dispensed well from a liquid type pump dispenser.

### Example 91

A composition was formulated with 0.48% sodium xylene sulphonate; 0.24% C9-11 ethoxylated alcohols; 0.008 2-propanol, 0.25% ethanol 2,2′-iminobis-N-(3-(branched decyloxy) propyl) derives. N oxides); 0.6% propylene glycol; and 0.2% PCMX with the remainder water. pH was about 6-6.5. This formulation foams very well.

### Example 92

A composition was formulated similar to that in example 90 with 2.7% sodium laureth sulfate, 0.6% lauryl glucoside (C10-16), lauryl glucoside (C6-12), 0.4% cocamidopropyl betaine, 0.19% ethanol, 0.32% PCMX, 0.8% sodium chloride, 1.25% cocamidopropyl PG-dimonium chloride phosphate, 0.41% hydrogenated starch hydrolysate, 0.6% glycerin, 0.2% ethylenediaminetetraacetic acid trisodium salt hydrate, 0.11% 1,3-dihydroxymethyl-5,5-dimethylhydantoin, 0.06% allantoin with 0.5% fragrance water soluble cucumber aloe and with citric acid for pH adjustment and with the remainder water. The final pH was 5.50. The viscosity at 23.9 C was 7 Zahn seconds with a G3 viscometer. This formulation was packaged in several types of foaming dispensers and was shown to dispense foam equally well from all.

This formulation, and others similar to it, were applied to fabrics to serve as pre-wet washing cloths. In particular nonwoven fabrics from the Dupont Sontara family of fabrics were useful for this application. For such products, packages in the form of individual or roll form wipes were particularly useful.

### Example 93

A composition was formulated with 7.2% sodium laureth sulfate, 1.3% lauryl glucoside (C10-16), lauryl glucoside (C6-12), 1.2% cocamidopropyl betaine, 0.19% ethanol, 0.32% PCMX, 0.8% sodium chloride, 1.25% cocamidopropyl PG-dimonium chloride phosphate, 0.41% hydrogenated starch hydrolysate, 0.6% glycerin, 0.2% ethylenediaminetetraacetic acid trisodium salt hydrate, 0.11% 1,3-dihydroxymethyl-5,5-dimethylhydantoin, 0.06% allantoin with 0.5% fragrance water soluble cucumber aloe and with citric acid for pH adjustment and with the remainder water. The final pH was 5.50. The viscosity at 23.3 C was 8 Zahn seconds with a G3 viscometer. This formulation was evaluated to achieve higher viscosities for dispensing in a liquid pump dispenser. Sodium chloride (NaCl) addition was used to increase the viscosity. At 0.249% NaCl the viscosity increased to 10.3 Zahn seconds, at 0.498% the viscosity increased to 20 Zahn seconds, at 0.621% the viscosity increased to 38 seconds, at 0.695% the viscosity increased to 58.3 Zahn seconds, at 0.745% the viscosity increased to 78.2 seconds, and at 0.794% the viscosity increased to 102 seconds. From this data it is very clear that there is a correlation of the % NaCl with Zahn viscosity and that the desired viscosity can be obtained by adjusting the sodium chloride concentration. At the latter sodium chloride concentration various types of liquid pumps (IRIS, Falcon, Flamingo, Euroflow, and Euroflow Contempo types as obtained from Kaufman Container Co.) were evaluated and shown to be acceptable.

### Example 94

A composition was formulated with 6.4% sodium laureth sulfate, 1.1% lauryl glucoside (C10-16), lauryl glucoside (C6-12), 1.1% cocamidopropyl betaine, no ethanol, 0.30% PCMX, 0.4% sodium chloride, 1.25% cocamidopropyl PG-dimonium chloride phosphate, 0.41% hydrogenated starch hydrolysate, 0.6% glycerin, 0.2% ethylenediaminetetraacetic acid trisodium salt hydrate, 0.11% 1,3-dihydroxymethyl-5,5-dimethylhydantoin, 0.06% allantoin with 0.5% fragrance water soluble cucumber aloe and with citric acid for pH adjustment and with the remainder water. The final pH was 5.50. The viscosity at 22.5 C was 8.33 Zahn seconds with a G3 viscometer.

Microbiological preservation assays were conducted on this formulation through an independent laboratory. The sample was evaluated to be free from contamination and adequately preserved. Time kill conducted on the samples showed Escherichia coli ATCC 8739 (baseline count of 1,090,000) was reduced 86.155% in 60 seconds and 88.655% in 120 seconds; Staphylococcus aureus ATCC 6538 (baseline count of 102,700) was reduced 86.280% in 60 seconds and 96.641% in 120 seconds; Pseudomonas aeruginosa ATCC 15442 (baseline count of 672,700) was reduced 97.770% in 60 seconds and 98.635% in 120 seconds; and Methicillin Resistant Staphylococcus aureus ATCC 33691 (MRSA) (baseline count of 654,500) was reduced 99.694% in 60 seconds and 99.756% in 120 seconds.

This formulation dispensed well from a liquid type pump dispenser.

### Example 95

A composition was formulated with 2.9% sodium laureth sulfate, 0.65% poly glucosides, 0.4% cocamidopropyl betaine, 0.8% propylene glycol, 0.4% PCMX, 1.25% cocamidopropyl PG-dimonium chloride phosphate, 0.41% hydrogenated starch hydrolysate, 0.6% glycerin, 0.2% ethylenediaminetetraacetic acid trisodium salt hydrate, 0.11% 1,3-dihydroxymethyl-5,5-dimethylhydantoin, 0.06% allantoin with 0.5% fragrance water soluble cucumber aloe and with citric acid for pH adjustment and with the remainder water. The final pH was 5.47. This formulation was packaged in a foaming dispenser and dispensed foam.

### Example 96

A composition was formulated with 2.9% sodium laureth sulfate, 0.65% poly glucosides, 0.4% cocamidopropyl betaine, 0.8% propylene glycol, 0.4% PCMX, 0.41% hydrogenated starch hydrolysate, 0.6% glycerin, 0.2% ethylenediaminetetraacetic acid trisodium salt hydrate, 0.11% 1,3-dihydroxymethyl-5,5-dimethylhydantoin, 0.06% allantoin with 0.5% fragrance water soluble cucumber aloe and with citric acid for pH adjustment and with the remainder water. The final pH was 5.50. This formulation was packaged in a foaming dispenser and dispensed nice foam.

### Example 97

A composition was formulated with 2.84% sodium laureth sulfate, 0.64% poly glucosides, 0.39% cocamidopropyl betaine, 0.78% propylene glycol, 0.39% PCMX, 0.40% hydrogenated starch hydrolysate, 0.59% glycerin, 0.20% ethylenediaminetetraacetic acid trisodium salt hydrate, 0.11% 1,3-dihydroxymethyl-5,5-dimethylhydantoin, 0.059% allantoin, 0.55% collagen hydrolyzate, 0.25% hydrolyzed quinoa with 0.49% fragrance water soluble cucumber aloe and with citric acid for pH adjustment and with the remainder water. The final pH was about 5.50. This formulation was packaged in a foaming dispenser and dispensed nice foam.

### Example 98

A composition was formulated with 4.42% sodium laureth sulfate, 1.14% poly glucosides, 0.4% cocamidopropyl betaine, 2.38% propylene glycol, 1.2% PCMX, 0.41% hydrogenated starch hydrolysate, 0.6% glycerin, 0.2% ethylenediaminetetraacetic acid trisodium salt hydrate, 0.11% 1,3-dihydroxymethyl-5,5-dimethylhydantoin, 0.06% allantoin, 0.55% collagen hydrolyzate, 0.25% hydrolyzed quinoa with 0.5% fragrance water soluble cucumber aloe and with citric acid for pH adjustment and with the remainder water. The final pH was 5.50. This formulation was packaged in a foaming dispenser and dispensed nice foam.

### Example 99

A composition was formulated with about 6.3% hydrogenated starch hydrolysates (CAS No. 68425-17-2, product Hystar CG from Corn Products) and 1.47% mixture of PEG-8 laurate, laureth-4, and chloroxylenol (PCMX) with resultant PCMX of about 0.25% (product Cola Mulse PCMX), with the remainder deionized water. Solution showed cloudiness and in time some layering. Hand testing showed that product had good feel.

### Example 100

A composition was formulated with about 0.25% benzethonium chloride (from Lonza), about 5.25% hydrogenated starch hydrolysates (CAS No. 68425-17-2, product Hystar CG from Corn Products), and 3.5% Dow Corning 193C fluid (PEG 12 dimethicone, silicone polyether copolymer) with the remainder deionized water. Solution was clear and goes on the hands well. After refrigeration for nearly 3 months solution was clear with no precipitates.

### Example 101

A composition was formulated with about 4.75% PVP-iodine and 2.1% sodium laureth sulfate and 0.4% lauryl glucoside and cocamidopropyl betaine (Surfacare APC, Surfactants) with the remainder deionized water. Solution is colored.

### Example 102

A composition was formulated with about 0.25% benzethonium chloride (from Lonza), about 4.2% hydrogenated starch hydrolysates (CAS No. 68425-17-2, product Hystar CG from Corn Products), 2.5% Dow Corning 193C fluid (PEG 12 dimethicone, silicone polyether copolymer), and 3% glycerin with the remainder deionized water. Solution goes on the hands well, dried readily, not sticky, but with some sheen and felt like leaving a coating.

### Example 103

A composition was formulated with about 0.25% benzethonium chloride (from Lonza), about 3.9% hydrogenated starch hydrolysates (CAS No. 68425-17-2, product Hystar CG from Corn Products), 2.5% Dow Corning 193C fluid (PEG 12 dimethicone, silicone polyether copolymer), and 3.5% glycerin with the remainder deionized water. Solution goes on the hands well and generally well accepted. It was incorporated in a sprayer.

### Example 104

A composition was formulated with about 0.25% benzethonium chloride (from Lonza), about 3.9% hydrogenated starch hydrolysates (CAS No. 68425-17-2, product Hystar CG from Corn Products), 2.5% Dow Corning 193C fluid (PEG 12 dimethicone, silicone polyether copolymer), 3.5% glycerin, and 0.25% allantoin with the remainder deionized water. Solution goes on the hands well and generally well accepted. To assess a fragrance a vanilla bean fragrance (Ungerer)/Tween 40 (1:1) solution added at about 1%.

### Example 105

A composition was formulated with about 0.25% benzethonium chloride (from Lonza), about 3.9% hydrogenated starch hydrolysates (CAS No. 68425-17-2, product Hystar CG from Corn Products), 2.5% Dow Corning 193C fluid (PEG 12 dimethicone, silicone polyether copolymer), 3.5% glycerin, and 0.5% Carbopol EDT 2020 with the remainder deionized water. pH of about 3.5 and adjusted up with sodium hydroxide to form a thick solution at pH of about 5.

### Example 106

A composition was formulated with about 0.25% benzethonium chloride (from Lonza), about 3.9% hydrogenated starch hydrolysates (CAS No. 68425-17-2, product Hystar CG from Corn Products), 2.5% Dow Corning 193C fluid (PEG 12 dimethicone, silicone polyether copolymer), 3.5% glycerin, and 0.3% Carbopol EDT 2020 with the remainder deionized water. pH of about 3.5 and adjusted up with sodium hydroxide to form a thick solution at about pH 5+.

### Example 107

A composition was formulated with about 0.25% benzethonium chloride (from Lonza), about 3.9% hydrogenated starch hydrolysates (CAS No. 68425-17-2, product Hystar CG from Corn Products), 2.5% Dow Corning 193C fluid (PEG 12 dimethicone, silicone polyether copolymer), 3.5% glycerin, and 0.25% Carbopol EDT 2020 with the remainder deionized water. pH of about 3.5-4 and adjusted up with sodium hydroxide and heating to form a thick solution at about pH of 6-6.5.

### Example 108

A composition was formulated with about 0.25% benzethonium chloride (from Lonza), about 3.9% hydrogenated starch hydrolysates (CAS No. 68425-17-2, product Hystar CG from Corn Products), 2.5% Dow Corning 193C fluid (PEG 12 dimethicone, silicone polyether copolymer), 3.5% glycerin, and 0.2% Carbopol EDT 2020 with the remainder deionized water. pH of about 5 and adjusted up with sodium hydroxide and heating to form a thick solution at about pH of 6+.

### Example 109

A composition was formulated with about 0.2% benzethonium chloride (from Lonza), about 3.9% hydrogenated starch hydrolysates (CAS No. 68425-17-2, product Hystar CG from Corn Products), 2.5% Dow Corning 193C fluid (PEG 12 dimethicone, silicone polyether copolymer), and 1% sodium pyrrolidone carboxylic acid (Macare PCA-50, Mason Chemical) with the remainder deionized water. Solution dries fast on hands but without a noted residual. Solution put into sprayer.

### Example 110

A composition was formulated with about 0.2% benzethonium chloride (from Lonza), about 3.9% hydrogenated starch hydrolysates (CAS No. 68425-17-2, product Hystar CG from Corn Products), 2.5% Dow Corning 193C fluid (PEG 12 dimethicone, silicone polyether copolymer), 3.5% glycerin, and 0.4% a fragrance a vanilla bean fragrance (Ungerer)/Tween 40 (1:1) with the remainder deionized water.

### Example 111

A composition was formulated with about 0.2% benzethonium chloride (from Lonza), about 3.9% hydrogenated starch hydrolysates (CAS No. 68425-17-2, product Hystar CG from Corn Products), 2.5% Dow Corning 193C fluid (PEG 12 dimethicone, silicone polyether copolymer), 3.5% glycerin, and 0.6% a fragrance a vanilla bean fragrance (Ungerer)/Tween 40 (1:2) with the remainder deionized water.

### Example 112

A composition was formulated with about 0.2% benzethonium chloride (from Lonza), about 3.9% hydrogenated starch hydrolysates (CAS No. 68425-17-2, product Hystar CG from Corn Products), 2.5% Dow Corning 193C fluid (PEG 12 dimethicone, silicone polyether copolymer), and 3.5% glycerin with the remainder deionized water. pH 5.12.

### Example 113

A composition was formulated with about 0.2% benzethonium chloride (from Lonza), about 3.9% hydrogenated starch hydrolysates (CAS No. 68425-17-2, product Hystar CG from Corn Products), 0.4% allantoin, 2.5% Dow Corning 193C fluid (PEG 12 dimethicone, silicone polyether copolymer), and 3.5% glycerin with the remainder deionized water. pH 3.96.

### Example 114

A composition was formulated with about 0.13% benzalkonium chloride (from Mason Chemical), about 3.9% hydrogenated starch hydrolysates (CAS No. 68425-17-2, product Hystar CG from Corn Products), 0.4% allantoin, 2.5% Dow Corning 193C fluid (PEG 12 dimethicone, silicone polyether copolymer), and 3.5% glycerin with the remainder deionized water. pH 4.05.

### Example 115

A composition formulated with about 0.13% benzalkonium chloride (from Mason Chemical) was used to assess various fragrances added at about 0.1%. Fragrances included were water soluble Febreze, lemon sugar, and Irish Spring all from Lebermuth. For some of these additions were made to wipes.

### Example 116

A composition was formulated with about 0.13% benzalkonium chloride (from Mason Chemical), about 3.9% hydrogenated starch hydrolysates (CAS No. 68425-17-2, product Hystar CG from Corn Products), 0.4% allantoin, 2.5% Dow Corning 193C fluid (PEG 12 dimethicone, silicone polyether copolymer), and 3.5% glycerin with the remainder deionized water. pH 4.18.

### Example 117

A composition was formulated with about 0.12% benzalkonium chloride (from Mason Chemical), 0.9% cetrimonium chloride, 0.12% dihydroxypropyl PEG-5 linoleaminium chloride, about 3.9% hydrogenated starch hydrolysates (CAS No. 68425-17-2, product Hystar CG from Corn Products), 0.4% allantoin, 2.5% Dow Corning 193C fluid (PEG 12 dimethicone, silicone polyether copolymer), and 3.5% glycerin with the remainder deionized water. pH 4.45. Put into foamer (Kaufmann), showed excellent foaming.

### Example 118

A composition was formulated with about 0.5% PCMX, about 0.9% sodium laureth sulfate, about 0.3% ethanol, about 0.1% sodium chloride, about 0.3% lauryl glucoside with about 0.1% fragrance (this mix is referred to as Surcide KG, Surfactants), about 3.9% hydrogenated starch hydrolysates (CAS No. 68425-17-2, product Hystar CG from Corn Products), 0.4% allantoin, 2.5% Dow Corning 193C fluid (PEG 12 dimethicone, silicone polyether copolymer), and 3.5% glycerin with the remainder deionized water. This mix had the fragrance of the Surcide KG. Put into foamer (Kaufmann), showed excellent foaming.

### Example 119

A composition was formulated with about 0.12% benzalkonium chloride (from Mason Chemical), 0.12% cetrimonium chloride, 0.12% dihydroxypropyl PEG-5 linoleaminium chloride, about 3.9% hydrogenated starch hydrolysates (CAS No. 68425-17-2, product Hystar CG from Corn Products), 0.4% allantoin, 2.5% Dow Corning 193C fluid (PEG 12 dimethicone, silicone polyether copolymer), and 3.5% glycerin with the remainder deionized water. pH 4.96. Put into foamers (Kaufmann) and spray bottles.

### Example 120

A composition was formulated with about 0.5% PCMX, about 1.5% propylene glycol, about 3.9% hydrogenated starch hydrolysates (CAS No. 68425-17-2, product Hystar CG from Corn Products), 0.4% allantoin, and 2.5% Dow Corning 193C fluid (PEG 12 dimethicone, silicone polyether copolymer) with the remainder deionized water.

### Example 121

A composition was formulated with about 0.5% PCMX, about 1.5% propylene glycol, about 3.9% hydrogenated starch hydrolysates (CAS No. 68425-17-2, product Hystar CG from Corn Products), 0.4% allantoin, and 2.5% Dow Corning 193C fluid (PEG 12 dimethicone, silicone polyether copolymer), and 1% Tween 40 with the remainder deionized water.

### Example 122

A lotion composition was formulated with about 3% PCMX, about 9% propylene glycol, 3% terpineol, 0.36% allantoin, 0.2% trisodium EDTA, 0.2% DMDMH, and 5% modified corn starch (Farmal MS 6892, Corn Products) with the remainder deionized water. pH about 6-6.5.

### Example 123

A lotion composition formulated by thickening EnviroSystem's EnviroTru. Added about 0.3% carbomer (Carbopol Ultrez 10, Lubrizol) to EnviroTru. pH about 4. Added potassium hydroxide to raise the pH to up to 8 to thicken solution.

### Example 124

A lotion composition formulated by thickening EnviroSystem's EcoTru. Added about 0.5% carbomer (Carbopol Ultrez 10, Lubrizol) to EcoTru. pH about 3.5-4. Added potassium/sodium hydroxide to raise the pH to up to 5.5 to thicken solution.

### Example 125

A lotion composition formulated by thickening EnviroSystem's EcoTru. Added about 0.6% carbomer (Carbopol Ultrez 10, Lubrizol) to EcoTru. pH about 3.5-4. Added potassium/sodium hydroxide to raise the pH to up to 5.5 to thicken solution.

### Example 126

A lotion composition formulated by thickening EnviroSystem's EnviroTru. Added about 0.6% carbomer (Carbopol Ultrez 10, Lubrizol) to EnviroTru. pH about 3.5-4. Added sodium hydroxide to raise the pH to up to 5.5 to thicken solution.

### Example 127

A lotion composition was formulated with about 3% PCMX, about 9% propylene glycol, 3% lemon mint (Lebermuth), 0.36% allantoin, 0.2% trisodium EDTA, 0.25% DMDMH, 1% glycerin, 5% modified corn starch (Farmal MS 6892, Corn Products), and 0.2% carbomer (Carbopol Ultrez 10, Lubrizol) with the remainder deionized water. pH about 4-4.5 and added sodium hydroxide to increase pH to about 5-5.5.

### Example 128

A composition was formulated with about 0.5% PCMX, about 0.9% sodium laureth sulfate, about 0.3% ethanol, about 0.1% sodium chloride, about 0.3% lauryl glucoside with about 0.1% fragrance (this mix is referred to as Surcide KG, Surfactants), about 3.9% hydrogenated starch hydrolysates (CAS No. 68425-17-2, product Hystar CG from Corn Products), 0.4% allantoin, 2.5% Dow Corning 193C fluid (PEG 12 dimethicone, silicone polyether copolymer), and 3.5% glycerin with the remainder deionized water. This mix had the fragrance of the Surcide KG. pH about 5.4. With refrigeration for about 49 days solution showed large crystals. With testing E. coli showed 52% and 45% reductions at 15 and 30 sec., S. aureus showed 43% and 33% reductions at 15 and 30 sec., and P. aeruginosa showed 67% and 31% reductions at 15 and 30 sec.

### Example 129

A composition was formulated with about 0.13% benzalkonium chloride, 0.9% cetrimonium chloride, 0.12% dihydroxypropyl PEG-5 linoleaminium chloride, 0.2% DMDMH, 0.2% trisodium EDTA, about 3.9% hydrogenated starch hydrolysates (CAS No. 68425-17-2, product Hystar CG from Corn Products), 0.4% allantoin, 2.5% Dow Corning 193C fluid (PEG 12 dimethicone, silicone polyether copolymer), and 3.5% glycerin with the remainder deionized water. pH about 5.6. With testing E. coli showed 99.999% reductions at 15 and 30 sec., S. aureus showed 99.92% and 99.98% reductions at 15 and 30 sec., and P. aeruginosa showed 99.999% reductions at 15 and 30 sec.

### Example 130

A composition was formulated with about 1% Antibac oil (blend of thyme white and carvacrol/thyme, wintergreen, eucalyptus, and menthol essential oils, Lebermuth), about 1% Tween 40, about 3.9% hydrogenated starch hydrolysates (CAS No. 68425-17-2, product Hystar CG from Corn Products), 0.4% allantoin, 2.5% Dow Corning 193C fluid (PEG 12 dimethicone, silicone polyether copolymer), and 3.5% glycerin with the remainder deionized water. pH about 5-5.5. Refrigerated sample had crystals.

### Example 131

A composition was formulated with about 1% Antibac oil (blend of thyme white and carvacrol essential oils, Lebermuth), about 1% Tomadol 91-6 (alcohols, C9-11, ethoxylated surfactant, Air Products), about 3.9% hydrogenated starch hydrolysates (CAS No. 68425-17-2, product Hystar CG from Corn Products), 0.4% allantoin, 2.5% Dow Corning 193C fluid (PEG 12 dimethicone, silicone polyether copolymer), and 3.5% glycerin with the remainder deionized water. pH about 4.2. From foamer there was some “watery” output.

### Example 132

A composition was formulated with about 1% Antibac oil (blend of thyme white and carvacrol essential oils, Lebermuth), about 1% Tween 40, about 3.9% hydrogenated starch hydrolysates (CAS No. 68425-17-2, product Hystar CG from Corn Products), 0.4% allantoin, 2.5% Dow Corning 193C fluid (PEG 12 dimethicone, silicone polyether copolymer), and 3.5% glycerin with the remainder deionized water. pH about 3.6. From foamer there was some “watery” output.

### Example 133

A composition was formulated with about 0.25% Antibac oil (blend of thyme white and carvacrol essential oils, Lebermuth), about 0.75% Tween 80, 0.9% cetrimonium chloride, 0.12% dihydroxypropyl PEG-5 linoleaminium chloride, about 3.9% hydrogenated starch hydrolysates (CAS No. 68425-17-2, product Hystar CG from Corn Products), 0.4% allantoin, and 3.5% glycerin with the remainder deionized water. pH about 4.1. Solution showed excellent foaming and after stored refrigerated for nearly one year was clear with no precipitates.

### Example 134

A composition was formulated with about 0.13% benzalkonium chloride, 0.12% cetrimonium chloride, 0.12% dihydroxypropyl PEG-5 linoleaminium chloride, 0.2% trisodium EDTA, about 3.9% hydrogenated starch hydrolysates (CAS No. 68425-17-2, product Hystar CG from Corn Products), 0.4% allantoin, and 3.5% glycerin with the remainder deionized water. pH was adjusted to about 5.1 with citric acid. Solution foamed very well.

### Example 135

A composition was formulated with about 0.25% Antibac oil (blend of thyme white and carvacrol essential oils, Lebermuth), about 0.75% Tomadol 91-6 (alcohols, C9-11, ethoxylated surfactant, Air Products), 0.12% cetrimonium chloride, 0.12% dihydroxypropyl PEG-5 linoleaminium chloride, 0.2% trisodium EDTA, about 3.9% hydrogenated starch hydrolysates (CAS No. 68425-17-2, product Hystar CG from Corn Products), 0.4% allantoin, and 3.5% glycerin with the remainder deionized water. pH was adjusted to about 5.1 with citric acid. Solution showed excellent foaming and when refrigerated over 8 months was clear and had no precipitates.

Some investigations were carried out on alcohol based sanitizers to improve foamability and provide comparisons with non-alcoholic based formulations. These are discussed in Examples 136-137.

### Example 136

A composition was formulated with about 62% ethanol, 0.25% Antibac oil (blend of thyme white and carvacrol essential oils, Lebermuth), 0.25% glycerin, and 10% foamer (Masurf AF-110DE, Mason Chemical Co., DEA-C8-18 perfluoroalkylethyl phosphate) with the remainder deionized water. pH was adjusted to about 4.8 with citric acid. Solution foamed very well from Kaufman foamer.

### Example 137

A composition was formulated with about 62% ethanol, 0.25% glycerin, and 10% foamer (Masurf AF-110DE, Mason Chemical Co., DEA-C8-18 perfluoroalkylethyl phosphate) with the remainder deionized water. pH was about 5.5. Solution foamed very well from Kaufman foamer.

### Example 138

A composition was formulated with about 1% Antibac oil (blend of thyme white and carvacrol essential oils, Lebermuth), about 3% Tomadol 91-6 (alcohols, C9-11, ethoxylated surfactant, Air Products), about 3.9% hydrogenated starch hydrolysates (CAS No. 68425-17-2, product Hystar CG from Corn Products), 0.4% allantoin, and 3.5% glycerin with the remainder deionized water. pH was adjusted to about 5.0 with citric acid. Solution showed excellent foaming and when refrigerated over 6 months was clear and had no precipitates.

### Example 139

A composition was formulated with about 0.33% thyme white oil, 0.33% carvacrol, 0.33% lemongrass essential oils (Lebermuth), about 3% Tomadol 91-6 (alcohols, C9-11, ethoxylated surfactant, Air Products), about 3.9% hydrogenated starch hydrolysates (CAS No. 68425-17-2, product Hystar CG from Corn Products), 0.4% allantoin, 0.2% trisodium EDTA, and 3.5% glycerin with the remainder deionized water. pH was adjusted to about 5.0 with citric acid.

### Example 140

A composition was formulated with about 0.33% thyme white oil, 0.33% carvacrol, 0.33% lemongrass essential oils (Lebermuth), about 3% Tween 80, about 3.9% hydrogenated starch hydrolysates (CAS No. 68425-17-2, product Hystar CG from Corn Products), 0.4% allantoin, 0.2% trisodium EDTA, and 3.5% glycerin with the remainder deionized water. pH was adjusted to about 5.0 with citric acid.

### Example 141

A composition was formulated with about 1% Antibac oil, about 3% Tween 80, 0.12% cetrimonium chloride, 0.12% dihydroxypropyl PEG-5 linoleaminium chloride, about 3.9% hydrogenated starch hydrolysates (CAS No. 68425-17-2, product Hystar CG from Corn Products), 0.4% allantoin, and 3.5% glycerin with the remainder deionized water. Solution was translucent. pH was about 4-4.5 without adjustment.

### Example 142

A composition was formulated with about 0.71% sodium laureth sulfate, about 0.24% decyl glucoside, about 0.25% PCMX, about 0.75% propylene glycol, about 3.9% hydrogenated starch hydrolysates (CAS No. 68425-17-2, product Hystar CG from Corn Products), about 0.4% allantoin, and about 3.5% glycerin with the remainder deionized water. Solution was clear. pH was about 5 without adjustment. Solution showed excellent foaming from Kaufman foamer, went on hands well, dried readily, and had no distinct fragrance. When refrigerated 19 days about a dozen crystals seen.

### Example 143

A composition was formulated with about 0.71% sodium laureth sulfate, about 0.24% decyl glucoside, about 0.25% PCMX, about 0.75% propylene glycol, about 3.9% hydrogenated starch hydrolysates (CAS No. 68425-17-2, product Hystar CG from Corn Products), about 0.4% allantoin, about 3.5% glycerin, and about 0.3% fragrance cucumber aloe (Belle Aire) with the remainder deionized water. Solution was clear. pH was about 5.5 without adjustment. Solution showed excellent foaming from Kaufman foamer, went on hands well, dried readily, and had no distinct fragrance. When refrigerated 19 days about 6 crystals seen.

### Example 144

A composition was formulated with about 0.71% sodium laureth sulfate, about 0.24% decyl glucoside, about 0.25% PCMX, about 0.75% propylene glycol, about 3.9% hydrogenated starch hydrolysates (CAS No. 68425-17-2, product Hystar CG from Corn Products), about 0.4% allantoin, about 4% glycerin, about 0.55% collagen hydrolysates, about 0.25% quinoa, and about 0.3% fragrance cucumber aloe (Belle Aire) with the remainder deionized water. Solution was clear. pH was about 5.5 without adjustment. Solution showed excellent foaming from Kaufman foamer, went on hands well, dried readily, and had a slight fragrance of the quinoa. When refrigerated 9 days no ppt., or crystals formed.

### Example 145

A composition which may be used as a hand and skin sanitizer was formulated with 1.0% solution of a mixture of the essential oils thyme, wintergreen, eucalyptus, and menthol, 0.4% allantoin, 5.5% of a commercial hydrogenated starch hydrolysate, 3.5% glycerin, 2.5% of a commercial silicone polyether PEG-12 dimethicone, and the remainder water. This formulation was shown to not completely solubilize the essential oils.

### Example 146

A composition of the type shown in Example 145 was made except the essential oil mixture was pre mixed about 1:1 with Tween 40 and added at 1% to a mixture of containing in the final mix of 0.4% allantoin, 5.5% of a commercial hydrogenated starch hydrolysate, 3.5% glycerin, 2.5% of a commercial silicone polyether PEG-12 dimethicone, and the remainder water. The final pH was about 4.0 and with citric acid adjusted to pH about 5.5.

### Example 147

A composition was made by mixing a pre-mix of essential oils in Tween 40 1:1 where the essential oils were equal quantities of thyme white, carvacrol, and cornmint and adding to a similar composition as in Example 146. This solution was not stable and the oils separated.

Note: The above examples 145-147 are given to demonstrate as other studies have also shown that mixtures of essential oils even with non-ionic surfactants are not necessarily stable and useful as some public literature would suggest. Studies were carried out also with anionic surfactants and various non-ionics including alcohol ethoxylates with varying types of essential oils in aqueous and alcoholic carriers and shown that unique solubility relationships exist and that properties such a foaming ability varies with the compositions.

### Example 148

An aqueous disinfecting, sanitizing, cleaning formulation was formulated with a mixture of essential oils. A 4:1 mix of Tween 40 and the essential oils lemongrass, carvacrol, and thyme white (5% total weight in formulation) was added to a ricinoleate solution (6% total ricinoleate weight in formulation) and the remainder made up in water. This solution served as a concentrate and was diluted to give a final concentration of about 0.2% total essential oils. The pH of this solution was adjusted to 8.5. This solution was applied in a spray to various surfaces (as tables, porcelain, counter tope) and shown to remove dirt and grime.

### Example 149

An aqueous disinfecting, sanitizing, cleaning formulation was formulated with a mixture of essential oils. A 4:1 mix of propylene glycol (a recognized GRAS chemistry) and the essential oils lemongrass, carvacrol, and thyme white (5% total weight in formulation) was added to a ricinoleate solution (6% total ricinoleate weight in formulation) and the remainder made up in water. This solution served as a concentrate and was diluted to give a final concentration of about 0.2% total essential oils. The pH of this solution was adjusted to 8.6. This solution was applied in a spray to various surfaces (as tables, porcelain, counter tops) and shown to remove dirt and grime.

### Example 150

An aqueous disinfecting, sanitizing, cleaning formulation was formulated with a mixture of essential oils. A 4:1 mix of Tween 80 and the essential oils lemongrass, carvacrol, and thyme white (5% total weight in formulation) was added to a ricinoleate solution (6% total ricinoleate weight in formulation) and the remainder made up in water. This solution served as a concentrate and was diluted to give a final concentration of about 0.2% total essential oils. The pH of this solution was adjusted to 8.5. This solution was applied in a spray to various surfaces (as tables, porcelain, counter tops) and shown to remove dirt and grime.

### Example 151

An aqueous disinfecting, sanitizing, cleaning formulation was formulated with a mixture of essential oils and the antimicrobial chloroxylenol (PCMX). A 4:1 mix of Tween 80 and the essential oils lemongrass, carvacrol, and thyme white (5% total weight in formulation) and PCMX (5% in formulation) was added to a ricinoleate solution (6% total ricinoleate weight in formulation) and the remainder made up in water. This solution served as a concentrate and was diluted to give a final concentration of about 0.2% total essential oils and 0.2% PCMX. The pH of this solution was 8.9.

### Example 152

An aqueous disinfecting, sanitizing, cleaning formulation was formulated with the essential oil lemongrass, 0.1%, Tween 80 0.2%, isopropyl alcohol 0.4%, 0.2% 1,3-dihydroxymethyl-5,5, dimethylhydantoin also known as DMDMH, 0.8% castor oil salts, and the remainder deionized water. The pH was adjusted to 8.5 with sodium hydroxide solution. The solution was added to polyester wipes (DuPont 8005) at a ratio of 4.5 times the weight of liquid to the weight of the wipes.

### Example 153

An aqueous disinfecting, sanitizing, cleaning formulation was formulated with the essential oil lemongrass, 0.1%, the commercial decyl glucoside Surfapon AG 42, isopropyl alcohol 0.4%, and the remainder deionized water. The pH was 9.6. The solution was added to polyester wipes (Dupont 8005), polypropylene wipes, and polyester-pulp at a ratio of 4.0 times the weight of liquid to the weight of the wipes. This solution in polyester wipes was evaluated for leaving a residual film on clear, transparent glass after wiping and allowed to dry and was shown to not leave any appreciable observable film.

### Example 154

An aqueous disinfecting, sanitizing, cleaning formulation was formulated with 0.1% of the essential oil lemongrass, 0.2% of the commercial lauramine oxide ColaLux LO, 0.4% of isopropyl alcohol, and the remainder deionized water. The pH was about 5.0. The solution was added to polyester wipes (Dupont 8005) at a ratio of about 4.0 times the weight of liquid to the weight of the wipes. This solution in polyester wipes was evaluated for leaving a residual film on clear, transparent glass after wiping and allowed to dry and was shown to not leave any appreciable observable film.

### Example 155

An aqueous disinfecting, sanitizing, cleaning formulation was formulated with 0.1% of the essential oil lemongrass, 0.2% of the commercial cocamide DEA ColaMid C, 0.4% of isopropyl alcohol, and the remainder deionized water. The solution was added to polyester wipes (Dupont 8005) at a ratio of about 4.0 times the weight of liquid to the weight of the wipes. This solution in polyester wipes was evaluated for leaving a residual film on clear, transparent glass after wiping and allowed to dry and was shown to not leave any appreciable observable film nearly as little as for Example 154.

### Example 156

An aqueous disinfecting, sanitizing, cleaning formulation was formulated with 0.1% of the essential oil lemongrass, 0.2% of the commercial lauramide DEA ColaMid AL, 0.4% of isopropyl alcohol, and the remainder deionized water. The solution was added to polyester wipes (Dupont 8005) at a ratio of about 4.0 times the weight of liquid to the weight of the wipes. This solution in polyester wipes was evaluated for leaving a residual film on clear, transparent glass after wiping and allowed to dry and was shown to not leave any appreciable observable film nearly as little as for Examples 154 and 155.

### Example 157

An aqueous formulation which may be used for treating an animate surface as skin was formulated with about 3% Tween 40, 1% mix of the essential oils thyme, wintergreen, eucalyptus, and menthol, 5.5% of a commercial hydrogenated starch hydrolysate, 3.6% glycerin, 2.5% of a commercial silicone polyether PEG-12 dimethicone, and the remainder water. Assessments of this formulation gave a particle size of 15.6 nm, zeta potential of −1.2 mV, conductivity of 0.09 mS/cm, and pH of about 4.0.

### Example 158

An alcoholic formulation which may be used for treating an animate surface as skin was formulated with about 62% ethanol, 0.1% glycerin, 1% mix of the essential oils thyme, wintergreen, eucalyptus, and menthol, and with increasing percentages up to 10% fluoroaliphatic phosphate (commercial alcohol foaming surfactant Masurf AF-110DE). This formulation foamed very well from a commercial personal foaming container.

### Example 159

An aqueous formulation which may be used for treating an animate surface as skin was formulated with about 0.75% Tween 80, 0.25% mix of the essential oils thyme, wintergreen, eucalyptus, and menthol, 0.4% allantoin, 5.5% of a commercial hydrogenated starch hydrolysate, 3.5% glycerin, 0.9% cetrimonium chloride, 0.12% dihydroxypropyl PEG-5 linoleaminium chloride, and the remainder water. This solution had a pH of 4.1 and showed excellent foaming from a commercial personal foaming container.

### Example 160

An alcoholic formulation which may be used for treating an animate surface as skin was formulated with about 62% ethanol, 0.25% glycerin, 0.25% mix of the essential oils thyme, wintergreen, eucalyptus, and menthol, and 10% fluoroaliphatic phosphate (commercial alcohol foaming surfactant Masurf AF-110DE). Citric acid was added to bring the pH to 4.6. This formulation foamed very well from a commercial personal foaming container.

### Example 161

An aqueous disinfecting, sanitizing, cleaning formulation was formulated with 0.2% of the essential oil lemongrass, 0.8% of Tween 80, 0.8% 2-dodecoxyethanol in Citrus Mulse, and the remainder deionized water. The pH was 8.41.

In the foregoing description, certain terms have been used for brevity, clarity and understanding, however, no unnecessary limitations are to be implied therefrom, because such terms are used for descriptive purposes and are intended to be broadly construed. Moreover, the descriptions and examples herein are by way of examples and the exemplary embodiment is not limited to the exact details shown and described.

In the following claims, any feature described as a means for performing a function shall be construed as encompassing any means known to those skilled in the art to be capable of performing the recited function, and shall not be limited to the features and structures shown herein or mere equivalents thereof. The description of the exemplary embodiment included in the Abstract included herewith shall not be deemed to limit the invention to features described therein.

Having described the features, discoveries and principles of the examples, the manner in which they are formulated and may be utilized, and the advantages and useful results attained; the new and useful compositions, ingredients, combinations, systems, operations, methods and relationships are set forth in the appended claims.

We claim:

1. A composition for disinfecting, sanitizing, and cleaning surfaces, comprising:
(a) water;
(b) at least one hydrophobic antimicrobial agent selected from the group consisting of (i) a halogen substituted xylenol compound, (ii) a phenolic compound, (iii) an antimicrobial natural or essential oil, (iv) an antimicrobial component from natural or essential oil, and (v) combinations of at least two of (i), (ii), (iii), and (iv);
(c) at least one surfactant, wherein said surfactant is selected from the group consisting of anionic surfactant, amphoteric surfactant, nonionic surfactant, and blends thereof;
(d) at least one solubilizing agent; and
(e) optionally at least one metal chelator; and
(f) optionally at least one thickening agent,
the composition having a particle size of less than 100 nm and a zeta potential number greater than −60 mv.

2. The composition of claim 1, wherein said thickening agent is present and is selected from the group consisting of sodium chloride, acrylic polymers, carbomers, polysaccharides, vegetable gums, proteins, and polyethylene glycol.

3. The composition of claim 1, wherein said antimicrobial agent is present at a level of from about 0.18 to about 0.28 weight percent.

4. The composition of claim 1 comprising:
(a) From about 97.78 to about 98.87 weight percent water;
(b) From about 0.2 to about 0.24 weight percent parachlorometaxylenol;
(c) From about 0.2 to about 0.4 weight percent potassium ricinoleate;
(d) From about 0.06 to about 0.1 weight percent sodium dodecyl sulfate;
(e) From about 0.2 to about 0.4 weight percent terpineol; and
(f) From about 0.03 to about 0.1 weight percent sodium polyphosphate;
(g) From about 0.3 to about 0.6 weight percent isopropyl alcohol;
(h) From about 0.04 to about 0.08 weight percent phenoxetol; and
(i) From about 0.1 to about 0.3 weight percent trisodium ethylene diamine tetraacetic acid.

5. The composition of claim 1 in the form of a thickened gel.

6. The composition of claim 1 wherein the antimicrobial component is a natural or essential oil.

7. A composition for disinfecting, sanitizing, and cleaning surfaces, comprising:
(a) water;
(b) at least one hydrophillic antimicrobial agent;
(c) at least one skin protectant;
(d) at least one foaming agent; and
(e) optionally, at least one thickening agent.

8. The composition of claim 7, wherein said hydrophillic antimicrobial agent is benzalkonium chloride.

9. The composition of claim 8, wherein said benzethonium chloride is present at a level of from 0.2 to 0.3 weight percent of the composition.

10. The composition of claim 7, wherein said skin protectant is hydrogenated starch hydrolysates.

11. The composition of claim 10, wherein said hydrogenated starch hydrolysates is present at a level of from 3.5 to 4.5 weight percent of the composition.

12. The composition of claim 7, wherein said foaming agent is a silicone polyether copolymer.

13. The composition of claim 12, wherein said silicone polyether copolymer is present at a level of from 2.0 to 3.0 weight percent of said composition.

14. A composition for disinfecting, sanitizing, and cleaning surfaces, comprising:
(a) water;
(b) at least one hydrophobic antimicrobial agent;
(c) at least one hydrophillic antimicrobial agent;
(d) at least one surfactant;
(e) at least one solubilizing agent;
(f) optionally at least one foaming agent;
(g) optionally at least one thickening agent.

15. The composition of claim 14, wherein said hydrophobic antimicrobial agent is at least one essential oil.

16. The composition of claim 14, wherein said hydrophillic antimicrobial agent is benzalkonium chloride.

17. The composition of claim 14, wherein said surfactant is a nonionic surfactant.

18. The composition of claim 14, wherein said solubilizing agent is selected from the group consisting of ethanol, isopropyl alcohol, and propylene glycol.

19. The composition of claim 14 applied to a wipe.

20. The composition of claim 19, wherein the composition was applied to said wipe at a level of 3 to 5 times the weight of said wipe.
